(19) United States
(12) Patent Application Publication
(10) Pub. No.: US 2012/0035251 A1
(43) Pub. Date: Feb. 9, 2012

(54) THIOPHENE INHIBITORS OF IKK-B SERINE-THREONINE PROTEIN KINASE

(75) Inventors: Alan Hastings Drummond, Oxfordshire (GB); Richard James Testar, Oxfordshire (GB)

(73) Assignee: CHROMA THERAPEUTICS LD., Oxfordshire (GB)

(21) Appl. No.: 13/265,848

(22) PCT Filed: Apr. 20, 2010
(86) PCT No.: PCT/GB2010/000790

§ 371 Date: Oct. 21, 2011

(30) Foreign Application Priority Data
Apr. 24, 2009 (GB) 0907120.0

(51) Int. Cl. A61K 31/381; A61P 35/00; A61P 37/00; A61P 21/00; A61P 3/10; A61P 25/28; A61P 17/00; C07D 333/36; A61P 1/00

(52) U.S. Cl. 514/447; 549/69

(57) ABSTRACT

The invention provides a compound which is (a) a thiophene carboxamide derivative of formula (IA) or (IB), or a tautomer thereof; or (b) a pharmaceutically acceptable salt, N-oxide, hydrate or solvate thereof, wherein L¹, A¹ and W are as defined herein. The compounds are useful as IKKβ inhibitors. The compounds can thus be used in medicine, for example in the treatment of autoimmune and inflammatory diseases.

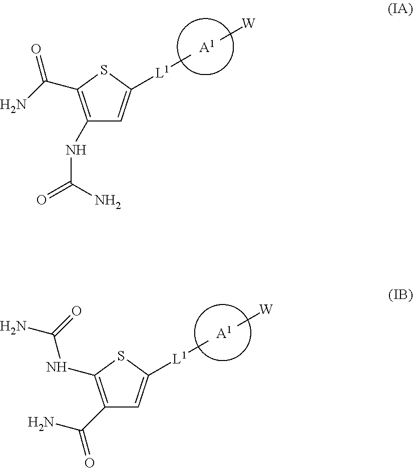

This invention relates to a series of amino acid derivatives, to compositions containing them, to processes for their preparation and to their use in medicine as IKK inhibitors.

### BACKGROUND TO THE INVENTION

The expression of many pro-inflammatory genes is regulated by the transcriptional activator nuclear factor-κB (NF-κB). Since their discovery these transcription factors have been suspected to play a pivotal role in chronic and acute inflammatory diseases. It now seems that aberrant regulation of NF-κB also underlies autoimmune diseases and different types of cancer.

Substances whose activity depends on the activation of NF-κB include: cytokines such as tumor necrosis factor TNF-α, interleukin (IL)-6, IL-8 and IL-1β; the adhesion molecules E-selectin, intercellular adhesion molecule (ICAM)-1 and vascular cell adhesion molecule (VCAM)-1; and the enzymes nitric oxide synthase (NOS) and cyclooxygenase (COX)-2. NF-κB normally resides in the cytoplasm of unstimulated cells as an inactive complex with a member of the IκB inhibitory protein family. However, upon cellular activation, IκB is phosphorylated by the IκB kinase (IKK) and is subsequently degraded. Free NF-κB then translocates to the nucleus where it mediates pro-inflammatory gene expression.

The three classical members of the IκB family are IκBα, IκBβ and IκBε. All of these require the phosphorylation of two key serine residues before they can be degraded. Two major enzymes IKK-α and IKK-βappear to be responsible for IκB phosphorylation. Dominant-negative (DN) versions of either of these enzymes (where ATP binding is disabled by the mutation of a key kinase domain residue) have been found to suppress the activation of NF-κB by TNF-α, IL-1β and LPS. Importantly IKK-β DN has been found to be a far more potent inhibitor than IKK-α DN (Zandi E, Cell, 1997, 91, 243). Furthermore, the generation of IKK-α and IKK-β deficient mice has established that IKK-β is required for activation of NF-κB by proinflammatory stimuli to occur and has corroborated biochemical data suggesting that IKK-β plays a dominant role in this pathway. Indeed it has been demonstrated that IKK-α is dispensable for NF-κB activation by these stimuli (Tanaka M, Immunity 1999, 10, 421).

Inhibition of IKK-β therefore represents a potentially attractive target for modulation of immune function and hence the development of drugs for the treatment of autoimmune diseases. For example, in relation to treatment of type II diabetes mellitus it has been shown that disruption of the NF-κB signalling pathways by methods such as tissue-specific deletion of IKK-β or pharmacological targetting of IKK-β can attenuate insulin resistance (Shoelson S et al., Gastroenterology 2007, 132, 2169).

A group of compounds has now been identified which are potent and selective inhibitors of IKK isoforms, particularly IKK-β. The compounds may thus be of use in medicine, for example in the treatment of a variety of proliferative disease states such as conditions related to the hyperactivity of IKK, as well as diseases modulated by the NF-κB cascade.

The general concept of conjugating an α-mono substituted glycine ester motif to a modulator of an intracellular enzyme or receptor, to obtain the benefits of intracellular accumulation of the carboxylic acid hydrolysis product is described in WO 2006/117567. However, this publication does not suggest that α,α-disubstituted glycine ester conjugates could be hydrolysed by intracellular carboxylesterases. It appears that the ability of the intracellular carboxyl esterases, principally hCE-1, hCE-2 and hCE-3, to hydrolyse α,α-disubstituted glycine ester has not previously been investigated.

### BRIEF DESCRIPTION OF THE INVENTION

The invention provides a compound which is: (a) a thiophene carboxamide derivative of formula (IA) or (IB), or a tautomer thereof; or (b) a pharmaceutically acceptable salt, N-oxide, hydrate or solvate thereof:

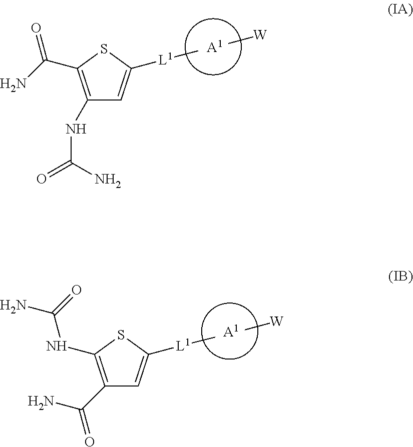

wherein:
L¹ represents a C₁₋₄ alkylene, C₂₋₄ alkenylene or C₂₋₄ alkynylene group, or L¹ represents a group —(CH₂)ₘ(C═O)NR¹ (CH₂)ₙ—, —(CH₂)ₘNR¹ (C═O)(CH₂)ₙ—, —(CH₂)ₘO(CH₂)ₙ—, —(CH₂)ₘS(CH₂)₁₁—, —(CH₂)ₘ(C═O)(CH₂)ₙ— or —(CH₂)ₘNR¹ (CH₂)ₙ—, in which R¹ is C₁₋₄ alkyl and m and n are the same or different and are 0, 1, 2, 3 or 4;
ring A¹ is a C₆₋₁₀ aryl, 5- to 10-membered heteroaryl, C₃₋₇ carbocyclyl or 5- to 10-membered heterocyclyl group which is optionally fused to a further C₆₋₁₀ aryl, 5- to 10-membered heteroaryl, C₃₋₇ carbocyclyl or 5- to 10-membered heterocyclyl group;
W represents a group of formula:

-L²-(Het)ₓ-Alk¹-R
wherein:
   L² represents a group -Alk²-, -Alk²-A²- or -Alk²-Alk³-;
   Alk² represents a bond or a C₁₋₄ alkylene, C₂₋₄ alkenylene or C₂₋₄ alkynylene group;
   Alk³ represents a C₁₋₄ alkylene, C₂₋₄ alkenylene or C₂₋₄ alkynylene group;
   A² represents a phenyl or 5- to 6-membered heteroaryl group which is unfused or fused to a further phenyl or 5- to 6-membered heteroaryl group;
   Het represents —O—, —S— or —NR′— where R′ represents hydrogen or unsubstituted C₁₋₂ alkyl;
   x is 0 or 1;
   Alk¹ represents a bond or a C₁₋₆ alkylene, C₂₋₆ alkenylene or C₂₋₆ alkynylene group, or a group -A³-Alk⁴- in which A³ represents a phenyl or 5- to 6-membered heteroaryl group which is unfused, or fused to a further phenyl or 5- to 6-membered heteroaryl group, and Alk⁴ represents a bond or a C₁₋₆ alkylene, C₂₋₆ alkenylene or C₂₋₆ alkynylene group;
   R represents a group of formula (X1), (X2), (Y1) or (Y2):

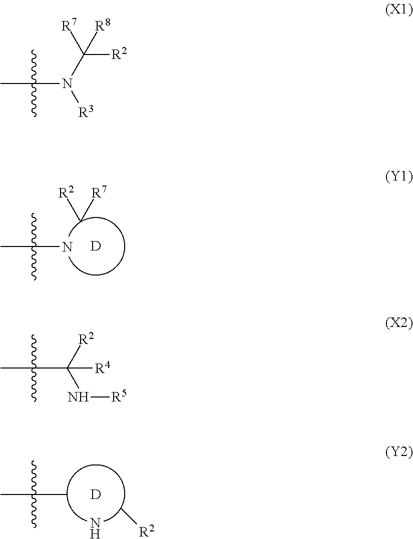

in which
R² is a group —COOH or an ester group which is hydrolysable by one or more intracellular carboxylesterase enzymes to a —COOH group;
R³ represents a hydrogen atom or a C₁₋₄ alkyl group;
R⁴, R⁷ and R⁸ are the same or different and each represents the α-substituent of a natural or non-natural α-amino acid, or R⁷ and R⁸, taken together with the carbon to which they are attached, form a 3- to 6-membered saturated Spiro cycloalkyl or heterocyclyl ring;
R⁵ represents a hydrogen atom or a C₁₋₆ alkyl, C₃₋₇ carbocyclyl, C₆₋₁₀ aryl or 5- to 6-membered heteroaryl group, or a group of formula —(C═O)R⁶, —(C═O)OR⁶, or —(C═O)NR⁶ wherein R⁶ is a hydrogen atom or a C₁₋₆ alkyl group; and
ring D is a 5- to 6-membered heterocyclyl group wherein: R² is linked to a ring carbon adjacent to the ring nitrogen shown; R⁷, if present, is linked to the same ring carbon as R²; and ring D is optionally fused to a second ring comprising a phenyl, 5- to 6-membered heteroaryl, C₃₋₇ carbocyclyl or 5- to 6-membered heterocyclyl group;
with the proviso that if R represents a group of formula (Y2) and the ring D is fused to a second ring comprising a phenyl, 5- to 6-membered heteroaryl, C₃₋₇ carbocyclyl or 5- to 6-membered heterocyclyl group then the bond shown intersected by a wavy line may be from a ring atom in ring D or said second ring;
   and wherein, unless otherwise stated:
any alkyl, alkenyl and alkynyl groups and moieties in R¹, R², R³, R⁴, R⁵, R⁶, R⁷, R⁸, L¹, Alk¹, Alk², Alk³ and Alk⁴ are the same or different and are each unsubstituted or substituted with 1, 2 or 3 unsubstituted substituents which are the same or different and are selected from halogen atoms and C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ haloalkoxy, C₂₋₄ haloalkenyloxy, hydroxyl, —SR′, cyano, nitro, C₁₋₄ hydroxyalkyl and —NR′R″ groups where R′ and R″ are the same or different and represent hydrogen or unsubstituted C₁₋₂ alkyl; and
any aryl, heteroaryl, carbocyclyl and heterocyclyl groups and moieties in A¹, A², A³, D and R⁵ are the same or different and are each unsubstituted or substituted by 1, 2, 3 or 4 unsubstituted substituents selected from halogen atoms, and cyano, nitro, C₁₋₄ alkyl, C₁₋₄ alkoxy, C₂₋₄ alkenyl, C₂₋₄ alkenyloxy, C₁₋₄ haloalkyl, C₂₋₄ haloalkenyl, C₁₋₄ haloalkoxy, C₂₋₄ haloalkenyloxy, hydroxyl, C₁₋₄ hydroxyalkyl, —SR′ and —NR′R″ groups wherein each R′ and R″ is the same or different and represents hydrogen or unsubstituted C₁₋₄ alkyl, or from substituents of formula —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —OCONH₂, —OCONHR^{A}, —OCONR^{A}R^{B}, —NHCOR^{A}, —NR^{B}COR^{A}, —NHCOOR^{A}, —NR^{B}COOR^{A}, —NR^{B}COOH, —NHCOOH, —NHSO₂R^{A}, —NR^{B}SO₂R^{A}, —NHSO₂OR^{A}, —NR^{B}SO₂OH, —NHSO₂H, —NR^{B}SO₂OR^{A}, —NHCONH₂, —NR^{A}CONH₂, —NHCONHR^{B}, —NR^{A}CONHR^{B}, —NHCONR^{A}R^{B} or —NR^{A}CONR^{A}R^{B} wherein R^{A} and R^{B} are the same or different and represent unsubstituted C₁₋₆ alkyl, C₃₋₆ cycloalkyl, non-fused phenyl or a non-fused 5- to 6-membered heteroaryl, or R^{A} and R^{B} when attached to the same nitrogen atom form a non-fused 5- or 6-membered heterocyclyl group.

The compounds of the invention contain an amino acid motif or an amino acid ester motif that is hydrolysable by an intracellular carboxylesterase. The compounds also contain a linker group, which separates the thiophene ring core of the molecule from the cyclic group carried on the side chain of the molecule that ultimately terminates in the amino acid or amino acid ester motif. Surprisingly, compounds of the invention having this combination of lipophilic amino acid or amino acid ester motif and linker group are potent and selective inhibitors of IKK isoforms, particular IKK-β.

Preferred compounds of the invention are those which contain an amino acid ester motif that is hydrolysable by an intracellular carboxylesterase. These compounds can easily cross a cell membrane and can then be hydrolysed to the acid by the intracellular carboxylesterases. The polar hydrolysis product accumulates in the cell since it does not cross the cell membrane as readily. Hence the IKK activity of the compound can be prolonged and enhanced within the cell. This accumulation effect may thus result in enhanced intracellular IKK activity of the compounds of the invention relative to their extracellular activity.

Furthermore, compounds of the invention where R is a group of formula (X1) or (Y1) have been found to accumulate selectively in monocytes. Thus, these compounds of the invention can advantageously be used where systemic administration is desired, since they are not very susceptible to pre-systemic metabolism and can thus reach target tissues in tact (whereupon they are converted inside target cells into the acid products). Preferential accumulation in monocyte cell lines can result in the IKK inhibitory effect of a dose of compound administered to a patient being concentrated on monocytes in preference to other neighbouring cells. This selectivity can therefore improve the therapeutic window associated with the compounds of the invention when used to target monocytic cells, for example in the treatment of cancer and autoimmune disease. Thus, for example, side effects associated with IKK inhibition of non-target, non-monocytic cells can be reduced.

Conversely, where local administration is desired it can be advantageous to use compounds of the invention where R is a group of formula (X2) or (Y2). Such compounds of the invention do not show monocyte selectivity and ester cleavage typically occurs more rapidly. This rapid rate of ester cleavage can be advantageous in reducing systemic exposure and consequent unwanted side effects.

Preferably the compounds of the invention are compounds of formula (IA) or (IB) or a tautomer thereof, or a pharmaceutically acceptable salt thereof.

The present invention also provides a compound as defined above for use in a method of treatment of the human or animal body.

The present invention further provides a pharmaceutical composition which comprises a compound as defined above and a pharmaceutically acceptable carrier or diluent.

In another aspect, the present invention provides a compound as defined above for use in the treatment of a disorder mediated by an IKK enzyme. The invention also provides use of a compound as defined above in the manufacture of a medicament for use in the treatment or prevention of a disorder mediated by an IKK enzyme. In these aspects, the IKK enzyme is preferably IKK-β.

Still further, the invention provides a method of treating or preventing a disorder mediated by IKK in a patient, which method comprises administering to said patient an effective amount of a compound as defined above.

### DETAILED DESCRIPTION OF THE INVENTION

Although the above definitions potentially include molecules of high molecular weight, it is preferable, in line with general principles of medicinal chemistry practice, that the compounds with which this invention is concerned should have molecular weights of no more than 600.

Preferably the alkyl, alkenyl and alkynyl groups and moieties in R¹, R², R³, R⁴, R⁵, R⁶, R⁷, R⁸, L¹, L², L³, Alk¹, Alk², Alk³ and Alk⁴ are unsubstituted or substituted with 1, 2 or 3, preferably 1 or 2, unsubstituted substituents which are the same or different and are selected from halogen, C₁₋₄ alkyl, C₂₋₄ alkenyl, C₁₋₄ alkoxy, hydroxyl, C₁₋₄ haloalkyl, C₂₋₄ haloalkenyl, C₁₋₄ haloalkyloxy and —NR′R″ wherein R′ and R″ are the same or different and represent hydrogen or C₁₋₂ alkyl. More preferred substituents are halogen, unsubstituted C₁₋₄ alkyl, C₁₋₄ alkoxy, hydroxyl and —NR′R″ groups where R′ and R″ are the same or different and represent hydrogen or unsubstituted C₁₋₂ alkyl. For example, particularly preferred substituents include unsubstituted C₁₋₄ alkyl, C₁₋₄ alkoxy, hydroxyl and —NR′R″ groups where R′ and R″ are the same or different and represent hydrogen or unsubstituted C₁₋₂ alkyl.

When the alkyl, alkylene, alkenylene and alkynylene moieties above are substituted by two or three substituents, it is preferred that not more than two substituents are selected from hydroxyl, cyano and nitro. More preferably, not more than one substituent is selected from hydroxyl, cyano and nitro.

As used herein, a C₁₋₆ alkyl group or moiety is a linear or branched alkyl group or moiety containing from 1 to 6 carbon atoms, for example a C₁₋₄ alkyl group or moiety containing from 1 to 4 carbon atoms. Examples of C₁₋₄ alkyl groups include methyl, ethyl, n-propyl, i-propyl, n-butyl, i-butyl and t-butyl. For the avoidance of doubt, where two alkyl moieties are present in a group, the alkyl moieties may be the same or different.

As used herein, a C₂₋₆ alkenyl group or moiety is a linear or branched alkenyl group or moiety having at least one double bond of either E or Z stereochemistry where applicable and containing from 2 to 6 carbon atoms, for example a C₂₋₄ alkenyl group or moiety containing from 2 to 4 carbon atoms, such as —CH═CH₂ or —CH₂—CH═CH₂, —CH₂—CH₂—CH═CH₂, —CH₂—CH═CH—CH₃, —CH═C(CH₃)—CH₃ and —CH₂—C(CH₃)═CH₂. For the avoidance of doubt, where two alkenyl moieties are present in a group, they may be the same or different.

As used herein, a C₂₋₆ alkynyl group or moiety is a linear or branched alkynyl group or moiety containing from 2 to 6 carbon atoms, for example a C₂₋₄ alkynyl group or moiety containing from 2 to 4 carbon atoms. Exemplary alkynyl groups include —C≡CH or —CH₂—C≡CH, as well as 1- and 2-butynyl, 2-methyl-2-propynyl, 2-pentynyl, 3-pentynyl, 4-pentynyl, 2-hexynyl, 3-hexynyl, 4-hexynyl and 5-hexynyl. For the avoidance of doubt, where two alkynyl moieties are present in a group, they may be the same or different.

As used herein, a C₁₋₆ alkylene group or moiety is a linear or branched alkylene group or moiety, for example a C₁₋₄ alkylene group or moiety. Examples include methylene, n-ethylene, n-propylene and —C(CH₃)₂— groups and moieties.

As used herein, a C₂₋₆ alkenylene group or moiety is a linear or branched alkenylene group or moiety, for example a C₂₋₄ alkenylene group or moiety. Examples include —CH═CH—, —CH═CH—CH₂—, —CH₂—CH═CH— and —CH═CH—CH═CH—.

As used herein, a C₂₋₆ alkynylene group or moiety is a linear or branched alkynylene group or moiety, for example a C₂₋₄ alkynylene group or moiety. Examples include —C═C—, —C═C—CH₂— and —CH₂—C═C—.

As used herein, a halogen atom is chlorine, fluorine, bromine or iodine.

As used herein, a C₁₋₆ alkoxy group or C₂₋₆ alkenyloxy group is typically a said C₁₋₆ alkyl (e.g. a C₁₋₄ alkyl) group or a said C₂₋₆ alkenyl (e.g. a C₂₋₄ alkenyl) group respectively which is attached to an oxygen atom.

A haloalkyl, haloalkenyl, haloalkoxy or haloalkenyloxy group is typically a said alkyl, alkenyl, alkoxy or alkenyloxy group respectively which is substituted by one or more said halogen atoms. Typically, it is substituted by 1, 2 or 3 said halogen atoms. Preferred haloalkyl and haloalkoxy groups include perhaloalkyl and perhaloalkoxy groups such as —CX₃ and —OCX₃ wherein X is a said halogen atom, for example chlorine and fluorine.

As used herein, a C₁₋₄ alkylthio or C₂₋₄ alkenylthio group is typically a said C₁₋₄ alkyl group or a C₂₋₄ alkenyl group respectively which is attached to a sulphur atom, for example —S—CH₃.

As used herein, a C₁₋₄ hydroxyalkyl group is a C₁₋₄ alkyl group substituted by one or more hydroxy groups. Typically, it is substituted by one, two or three hydroxy groups. Preferably, it is substituted by a single hydroxy group.

As used herein, a C₆₋₁₀ aryl group or moiety is a monocyclic, 6- to 10-membered aromatic hydrocarbon ring having from 6 to 10 carbon atoms. Phenyl is preferred.

Preferably a C₆₋₁₀ aryl group or moiety, as used herein, is unfused. However, when a C₆₋₁₀ aryl group or moiety is fused to a further C₆₋₁₀ aryl, 5- to 10-membered heterocyclyl, C₃₋₇ carbocyclyl or 5- to 10-membered heterocyclyl group, it is preferably fused to a further phenyl, 5- to 6-membered heterocyclyl, C₃₋₇ carbocyclyl or 5- to 6-membered heterocyclyl group, more preferably to a 5- to 6-membered heteroaryl or 5- to 6-membered heterocyclyl group. Most preferably it is fused to a 5- to 6-membered heterocyclyl group. In this case, preferred 5- to 6-membered heterocyclyl groups include tetrahydrofuranyl, tetrahydrothienyl, pyrrolidinyl, dithiolanyl, dioxolanyl, oxazolidinyl, imidazolyl, isoxazolidinyl, imidazolidinyl, pyrazolidinyl, thioxolanyl, thiazolidinyl and isothiazolidinyl, more preferably oxazolidinyl, imidazolidinyl, thiazolidinyl, thioxolanyl, dioxolanyl and dithiolanyl, most preferably dioxolanyl.

As used herein, a 5- to 10-membered heteroaryl group or moiety is a monocyclic 5- to 10-membered aromatic ring, such as a 5- or 6-membered ring, containing at least one heteroatom, for example 1, 2, 3 or 4 heteroatoms, selected from O, S and N. When the ring contains 4 heteroatoms these are preferably all nitrogen atoms. Examples include thienyl, furyl, pyrrolyl, imidazolyl, thiazolyl, isothiazolyl, pyrazolyl, oxazolyl, isoxazolyl, triazolyl, thiadiazolyl, oxadiazolyl, pyridinyl, pyridazinyl, pyrimidinyl, pyrazinyl, triazinyl and tetrazolyl groups. Thienyl, pyrrolyl, imidazolyl, thiazolyl, isothiazolyl, pyrazolyl, oxazolyl, isoxazolyl, triazolyl, pyridinyl, pyridazinyl, pyrimidinyl and pyrazinyl groups are preferred, e.g. pyrrolyl, imidazolyl, thiazolyl, isothiazolyl, pyrazolyl, oxazolyl, isoxazolyl, triazolyl, pyridinyl, pyridazinyl, pyrimidinyl and pyrazinyl groups. More preferred groups are thienyl, pyridinyl, pyridazinyl, pyrimidinyl, pyrazinyl, pyrrolyl and triazinyl, e.g. pyridinyl, pyridazinyl, pyrimidinyl, pyrazinyl, pyrrolyl and triazinyl, most preferably pyridinyl.

Preferably a heteroaryl group or moiety, as used herein, is unfused. However, when a heteroaryl group or moiety is fused to another group, it may be fused to a further phenyl, 5- to 10-membered heteroaryl, 5- to 10-membered heterocyclyl or C₃₋₇ carbocyclyl group. Preferably it is preferably fused to a phenyl, 5- to 6-membered heteroaryl or 5- to 6-membered heterocyclyl ring, more preferably it is fused to a phenyl group. Examples include benzothienyl, benzofuryl, benzimidazolyl, benzothiazolyl, benzisothiazolyl, benzoxazolyl, benzisoxazolyl, benztriazolyl, indolyl, isoindolyl and indazolyl groups. Preferred groups include indolyl, isoindolyl, benzimidazolyl, indazolyl, benzofuryl, benzothienyl, benzoxazolyl, benzisoxazolyl, benzothiazolyl and benzisothiazolyl groups, more preferably benzimidazolyl, benzoxazolyl and benzothiazolyl, most preferably benzothiazolyl.

As used herein, a 5- to 10-membered heterocyclyl group or moiety is a non-aromatic, saturated or unsaturated C₅₋₁₀ carbocyclic ring in which one or more, for example 1, 2, 3 or 4, of the carbon atoms are replaced with a moiety selected from N, O, S, S(O) and S(O)₂, and wherein one or more of the remaining carbon atoms is optionally replaced by a group —C(O)— or —C(S)—. When one or more of the remaining carbon atoms is replaced by a group —C(O)— or —C(S)—, preferably only one or two (more preferably two) such carbon atoms are replaced. Typically, the 5- to 10-membered heterocyclyl ring is a 5- to 6-membered ring.

Suitable heterocyclyl groups and moieties include azetidinyl, oxetanyl, thietanyl, pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, methylenedioxyphenyl, ethylenedioxyphenyl, thiomorpholinyl, S-oxo-thiomorpholinyl, S,S-dioxo-thiomorpholinyl, morpholinyl, 1,3-dioxolanyl, 1,4-dioxolanyl, trioxolanyl, trithianyl, imidazolinyl, pyranyl, pyrazolinyl, thioxolanyl, thioxothiazolidinyl, 1H-pyrazol-5-(4H)-onyl, 1,3,4-thiadiazol-2(3H)-thionyl, oxopyrrolidinyl, oxothiazolidinyl, oxopyrazolidinyl, succinimido and maleimido groups and moieties. Preferred heterocyclyl groups are pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, thiomorpholinyl and morpholinyl groups and moieties. More preferred heterocyclyl groups are tetrahydropyranyl, tetrahydrothiopyranyl, thiomorpholinyl, tetrahydrofuranyl, tetrahydrothienyl, piperidinyl, morpholinyl and pyrrolidinyl groups, and variants where one or two ring carbon atoms are replaced with —C(O)— groups. Particularly preferred groups include tetrahydrofuranyl and pyrrolyl-2,5-dione.

When a heterocyclyl group or moiety is fused to another group, it may be fused to a further phenyl, 5- to 10-membered heteroaryl, 5- to 10-membered heterocyclyl or C₃₋₇ carbocyclyl group, more preferably to a further phenyl, 5- to 6-membered heteroaryl or 5- to 6-membered heterocyclyl group. Preferably it is monocyclic (i.e. it is unfused).

For the avoidance of doubt, although the above definitions of heteroaryl and heterocyclyl groups refer to an “N” moiety which can be present in the ring, as will be evident to a skilled chemist the N atom will be protonated (or will carry a substituent as defined below) if it is attached to each of the adjacent ring atoms via a single bond.

As used herein, a C₃₋₇ carbocyclic group or moiety is a non-aromatic saturated or unsaturated hydrocarbon ring having from 3 to 7 carbon atoms. Preferably it is a saturated or mono-unsaturated hydrocarbon ring (i.e. a cycloalkyl moiety or a cycloalkenyl moiety) having from 3 to 7 carbon atoms, more preferably having from 3 to 6 carbon atoms. Examples include cyclopropyl, cyclobutyl, cyclopentyl and cyclohexyl and their mono-unsaturated variants, more particularly cyclopentyl and cyclohexyl. A C₃₋₇ carbocyclyl group or moiety also includes C₃₋₇ carbocyclyl groups or moieties described above but wherein one or more ring carbon atoms are replaced by a group —C(O)—. More preferably, 0, 1 or 2 ring carbon atoms (most preferably 0 or 2) are replaced by —C(O)—. A preferred such group is benzoquinone.

When a carbocyclyl group or moiety is fused to another group, it may be fused to a further phenyl, 5- to 10-membered heteroaryl, 5- to 10-membered heterocyclyl or C₃₋₇ carbocyclyl group, more preferably to a further phenyl, 5- to 6-membered heteroaryl or 5- to 6-membered heterocyclyl ring. For example it may be fused to a further phenyl ring. An exemplary fused carbocyclyl group is indanyl. More preferably carbocyclyl groups are monocyclic (i.e. non-fused).

When the aryl, heteroaryl, heterocyclyl and carbocyclyl moieties in A¹, A², A³, B, D and R⁵ are substituted by two, three or four substituents, it is preferred that not more than two substituents are selected from hydroxyl, cyano and nitro. More preferably, not more than one substituent is selected from hydroxyl, cyano and nitro. Furthermore, when the aryl, heteroaryl, heterocyclyl and carbocyclyl moieties are substituted by two or three substituents, it is preferred that not more than one substituent is selected from —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —OCONH₂, —OCONHR^{A}, —OCONR^{A}R^{B}, —NHCOR^{A}, —NR^{B}COR^{A}, —NHCOOR^{A}, —NR^{B}COOR^{A}, —NR^{B}COOH, —NHCOOH, —NHSO₂R^{A}, —NR^{B}SO₂R^{A}, —NHSO₂OR^{A}, —NR^{B}SO₂OH, —NHSO₂H, —NR^{B}SO₂OR^{A}, —NHCONH₂, —NR^{A}CONH₂, —NHCONHR^{B}, —NR^{A}CONHR^{B}, —NHCONR^{A}R^{B} or —NR^{A}CONR^{A}R^{B}.

Typically the phenyl, heteroaryl, heterocyclyl and carbocyclyl moieties in the aryl, heteroaryl, carbocyclyl and heterocyclyl groups and moieties in A¹, A², A³, B, D and R⁵ are unsubstituted or substituted by 1, 2, 3 or 4 substituents, for example by 1, 2 or 3 substituents. Preferred substituents include halogen atoms and C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ haloalkoxy, C₂₋₄ haloalkenyloxy, hydroxyl, mercapto, cyano, nitro, C₁₋₄ hydroxyalkyl, C₂₋₄ hydroxyalkenyl, C₁₋₄ alkylthio, C₂₋₄ alkenylthio and —NR′R″ groups wherein each R′ and R″ is the same or different and represents hydrogen or C₁₋₄ alkyl. Preferably the substituents are themselves unsubstituted. More preferred substituents include halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups where R′ and R″ are the same or different and represent hydrogen or unsubstituted C₁₋₂ alkyl. More preferred substituents include halogen atoms and C₁₋₂ alkyl and C₁₋₂ alkoxy groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (IA) or (IB) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like.

Compounds of the invention which contain one or more actual or potential chiral centres, because of the presence of asymmetric carbon atoms, can exist as a number of diastereoisomers with R or S stereochemistry at each chiral centre. The invention includes all such diastereoisomers and mixtures thereof.

For the avoidance of doubt, where L¹ represents a group —(CH₂)ₘ(C═O)NR¹ (CH₂)ₙ—, —(CH₂)ₘNR¹(C═O)(CH₂)ₙ—, —(CH₂)ₘO(CH₂)ₙ—, —(CH₂)ₘS(CH₂)ₙ—, —(CH₂)ₘ(C═O)(CH₂)ₙ— or —(CH₂)ₘNR¹(CH₂)ₙ—, the left hand side of that group as drawn is attached to the thiophene ring moiety of the compounds of the invention and the right hand side of that group is thus attached to the ring A¹ of the compounds of the invention. Preferably, the sum of the integers n and m is not greater than 6. More preferably the sum of the integers n and m is not greater than 4. Most preferably the sum of the integers n and m is 3 or less. Preferably, at least one of m and n is equal to zero. More preferably at least m is equal to zero.

In a preferred embodiment of the invention, L¹ is not a C₂₋₄ alkynylene group.

Preferably L¹ represents —O—, —S—, —NR¹— or C₁₋₄ alkylene, which C₁₋₄ alkylene is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups wherein R′ and R″ are the same or different and represent hydrogen or unsubstituted C₁₋₂ alkyl, and wherein R¹ represents unsubstituted C₁₋₄ alkyl.

More preferably L¹ represents —O— or C₁₋₄ alkylene wherein the C₁₋₄ alkylene moiety is unsubstituted or substituted with 1 or 2 unsubstituted substituents which are the same or different and are selected from halogen atoms and C₁₋₂ alkoxy, hydroxyl, C₁₋₂ haloalkyl and —NR′R″ groups wherein R′ and R″ are the same or different and represent hydrogen or unsubstituted C₁₋₂ alkyl. More preferably still, L¹ represents C₁₋₄ alkylene which is unsubstituted or substituted with 1 or 2 unsubstituted substituents which are the same or different and are selected from halogen atoms and C₁₋₂ alkoxy and C₁₋₂ haloalkyl groups

Most preferably L¹ represents unsubstituted C₁₋₄ alkylene, for example methyl, ethyl, n-propyl or i-propyl. In a particularly preferred embodiment L¹ represents methyl.

Preferably A¹ represents a phenyl, 5- to 6-membered heteroaryl, C₃₋₇ carbocyclyl or 5- to 6-membered heterocyclyl group which is unfused or fused to a further phenyl, 5- to 6-membered heteroaryl, C₃₋₇ carbocyclyl or 5- to 6-membered heterocyclyl group.

More preferably A¹ represents a phenyl or 5- to 6-membered heteroaryl group which is unfused or fused to a further phenyl or 5- to 6-membered heterocyclyl group. When A¹ represents a phenyl or 5- to 6-membered heteroaryl group which is unfused or fused to a 5- to 6-membered heterocyclyl group, the heterocyclyl group is preferably a dioxole group. For example, when A¹ represents a phenyl or 5- to 6-membered heteroaryl group which is unfused or fused to a 5- to 6-membered heterocyclyl group, a preferred A¹ group is benzodioxole.

More preferably A¹ represents a phenyl or 5- to 6-membered heteroaryl group which is unfused or fused to a further phenyl group. More preferably A¹ represents an unfused phenyl or 5- to 6-membered heteroaryl group, more preferably an unfused phenyl group such as a 1,4-phenylene or 1,3-phenylene group. In one preferred embodiment, A¹ represents a 1,3-phenylene group.

Preferably the A¹ group is unsubstituted or bears 1, 2 or 3 substituents. Where more than one substituent is present the substituents may be the same or different. Where more than one substituent is present preferably only one substituent is a hydroxyl, cyano or nitro group.

Preferred substituents on A¹ are selected from halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups where R′ and R″ are the same or different and represent hydrogen or unsubstituted C₁₋₂ alkyl.

More preferred substituents on A¹ are selected from halogen atoms and unsubstituted C₁₋₄ alkyl, C₁₋₄ alkoxy, hydroxyl and —NR′R″ groups where R′ and R″ are the same or different and represent hydrogen or unsubstituted C₁₋₂ alkyl. More preferably still the substituents on A¹ are selected from halogen atoms and unsubstituted C₁₋₂ alkyl and C₁₋₂ alkoxy groups.

Most preferably the A¹ group is unsubstituted.

Preferably Alk² represents a bond or a C₁₋₃ alkylene, C₂₋₃ alkenylene or C₂₋₃ alkynylene group. More preferably Alk² represents a C₁₋₃ alkylene, C₂₋₃ alkenylene or C₂₋₃ alkynylene group.

Preferably the Alk² group is unsubstituted or substituted with 1, 2 or 3 unsubstituted substituents selected from halogen atoms, and C₁₋₄ alkoxy, hydroxyl, C₁₋₄ haloalkyl, C₂₋₄ haloalkenyl, C₁₋₄ haloalkoxy and —NR′R″ groups where R′ and R″ are the same or different and represent hydrogen or unsubstituted C₁₋₂ alkyl. More preferably the Alk² group is unsubstituted or substituted with 1, 2 or 3, more preferably 1 or 2, unsubstituted substituents selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl. Most preferably the Alk² group is unsubstituted.

More preferably Alk² represents an unsubstituted methylene, ethylene (—CH₂CH₂—), vinylene (—CH═CH—) or ethynylene (—C≡C—) group. Most preferably Alk² represents a C₁₋₂ alkylene group, such as methylene.

When L² represents -Alk²-A²-, preferably A² represents an unfused phenyl or unfused 5- to 6-membered heteroaryl group. More preferably A² represents an unfused phenyl group. The Alk² and Het or Alk¹ groups can be attached to the phenyl group at any position, although it is preferred that the Alk² and Het or Alk¹ groups are attached in a meta- or para-relationship to one another, more preferably in a para-relationship.

Preferably the A² group bears 0, 1, 2 or 3 substituents, more preferably 0, 1 or 2 substituents. Where more than one substituent is present the substituents may be the same or different. Where more than one substituent is present preferably only one substituent is a hydroxyl, cyano or nitro group.

Preferred substituents on A² are selected from halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups where R′ and R″ are the same or different and represent hydrogen or unsubstituted C₁₋₂ alkyl.

More preferred substituents on A² are selected from halogen atoms and unsubstituted C₁₋₄ alkyl, C₁₋₄ alkoxy, hydroxyl and —NR′R″ groups where R′ and R″ are the same or different and represent hydrogen or unsubstituted C₁₋₂ alkyl.

More preferably the substituents on A² are selected from halogen atoms and unsubstituted C₁₋₂ alkyl and C₁₋₂ alkoxy groups. Most preferably the A² group is unsubstituted.

When L² represents -Alk²-Alk³, preferably Alk³ represents a C₁₋₄ alkylene, C₂₋₄ alkenylene or C₂₋₄ alkynylene group which is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl. More preferably, Alk³ represents an unsubstituted C₁₋₄ alkylene, C₂₋₄ alkenylene or C₂₋₄ alkynylene group. Most preferably Alk³ represents an unsubstituted C₁₋₄ alkylene, for example a C₃₋₄ alkylene group, more preferably a group —CH₂—CH₂—CH₂—.

Preferably L² is -Alk²-.

When present, the Het group preferably represents —O—, —NR′ or —S—, wherein R′ represents hydrogen or unsubstituted methyl. More preferably, the Het group represents —O—, —NH or —S—. Most preferably, the Het group is —O—.

When L² is -Alk²- preferably x is 0. In another preferred embodiment, x is 0.

Preferably Alk¹ represents a bond or a C₁₋₆ alkylene group or a group -A³-Alk⁴-. More preferably Alk¹ represents a bond or a C₁₋₆ alkylene group. Most preferably Alk¹ represents a bond.

When Alk¹ represents a C₁₋₆ alkylene group, it is preferably a C₁₋₄ alkylene group, more preferably a C₁₋₃ alkylene group, preferably a methylene or propylene group.

When Alk¹ represents a C₁₋₆ alkylene group preferably the Alk¹ group is unsubstituted or substituted with 1, 2 or 3 unsubstituted substituents selected from halogen atoms and C₁₋₄ alkoxy, hydroxyl, C₁₋₄ haloalkyl, C₂₋₄ haloalkenyl, C₁₋₄ haloalkoxy and —NR′R″ groups where R′ and R″ are the same or different and represent hydrogen or unsubstituted C₁₋₂ alkyl. More preferably the Alk¹ group is unsubstituted or substituted with 1 or 2, more preferably 1, unsubstituted substituent selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl. Most preferably Alk¹ is unsubstituted.

When Alk¹ represents a group -A³-Alk⁴- then A³ preferably represents an unfused phenyl or unfused 5- to 6-membered heteroaryl group which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″ groups where R′ and R″ are the same or different and represent hydrogen or unsubstituted C₁₋₂ alkyl, and Alk⁴ represents a bond or an C₁₋₃ alkylene, C₂₋₃ alkenylene or C₂₋₃ alkynylene group which is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl.

When Alk¹ represents a group -A³-Alk⁴-, preferably A³ represents an unfused phenyl or unfused 5- to 6-membered heteroaryl group which is unsubstituted or substituted with 1, 2 or 3 substituents selected from halogen atoms and unsubstituted C₁₋₄ alkyl, C₁₋₄ alkoxy, hydroxyl and —NR′R″ groups where R′ and R″ are the same or different and represent hydrogen or unsubstituted C₁₋₂ alkyl. More preferably A³ represents an unfused phenyl which is unsubstituted or substituted with 1, 2 or 3 substituents selected from halogen atoms and unsubstituted C₁₋₄ alkyl, C₁₋₄ alkoxy, hydroxyl and —NR′R″ groups where R′ and R″ are the same or different and represent hydrogen or unsubstituted C₁₋₂ alkyl. Most preferably A³ represents an unsubstituted, unfused phenyl group.

When Alk¹ represents a group -A³-Alk⁴-, preferably Alk⁴ represents an unsubstituted C₁₋₆ alkylene group, more preferably an unsubstituted C₁₋₃ alkylene group and more preferably still an unsubstituted C₁₋₂ alkylene group, in particular a methylene group —CH₂— or an ethylene group —CH₂—CH₂—.

Preferably only one of A² and A³ is present, i.e. it is preferred that L² is not -Alk²-A²- when Alk¹ is -A³-Alk⁴- and vice versa.

When x is 1, preferably Alk¹ is a C₃ alkylene group. When L² is -Alk²-, preferably x is 0 and Alk¹ is a bond or a C₃ alkylene group, for example a C₃ alkylene group. When L² is -Alk²-A²-, preferably x is 1 and Alk¹ is a C₁ alkylene group.

Preferred groups (Y1) and (Y2) include those where Ring D is a non-fused 5- to 6-membered heteroaryl or heterocyclyl group where R² is linked to a carbon atom adjacent the nitrogen atom shown in Ring D. More preferably Ring D is a non-fused 5- to 6-membered heterocyclyl group, for example a pyrrolidinyl, oxazolidinyl, isoxazolidinyl, imidazolidinyl, pyrazolidinyl, thiazolidinyl, isothiazolidinyl, piperidinyl, hexahydropyrimidinyl, piperazinyl, morpholinyl or thiomorpholinyl group. More preferably Ring D is a pyrrolidinyl, piperazinyl or piperidinyl group, more preferably a piperidyl or piperazinyl group.

When the group R is of formula (Y1), ring D, in addition to bearing groups R² and R⁷, is preferably unsubstituted or substituted by 1 or 2 groups selected from halogen atoms and C₁₋₄ alkyl, C₁₋₄ alkoxy and hydroxyl groups. More preferably Ring D, apart from bearing the groups R² and R⁷, is unsubstituted.

When the group R is of formula (Y2), ring D, in addition to bearing the group R², is preferably unsubstituted or substituted by 1 or 2 groups selected from halogen atoms and C₁₋₄ alkyl, C₁₋₄ alkoxy and hydroxyl groups. More preferably Ring D, apart from bearing the group R², is unsubstituted.

When R is a group of formula (Y2), particularly preferred Ring D groups are:

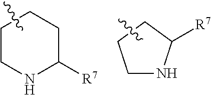

When R represents a group of formula (X1), R³ preferably represents a hydrogen atom or an unsubstituted C₁₋₂ alkyl. More preferably R³ represents a hydrogen atom or an unsubstituted methyl. Most preferably R³ represents a hydrogen atom.

When R represents a group of formula (X2), R⁵ preferably represents a hydrogen atom or an unsubstituted C₁₋₄ alkyl group, or a group of formula —(C═O)R⁶, —(C═O)OR⁶, or —(C═O)NR¹² wherein R⁶ is a hydrogen atom or an unsubstituted C₁₋₄ alkyl group. More preferably R⁵ represents a hydrogen atom or an unsubstituted C₁₋₂ alkyl group. Most preferably R⁵ represents a hydrogen atom.

For the avoidance of doubt, where R represents a group of formula (X1), the groups R⁷ and R⁸ may be the same or different.

When the substituents R⁷ and R⁸, taken together with the carbon to which they are attached, form a 3- to 6-membered saturated Spiro cycloalkyl or heterocyclyl ring, suitable Spiro cycloalkyl rings include cyclopropyl, cyclopentyl and cyclohexyl rings while suitable spiro heterocyclyl rings include piperidin-4-yl rings.

For the avoidance of doubt in the definition of the groups R⁴, R⁷ and R⁸ as “the α-substituent of a natural or non-natural α-amino acid”, “α-amino acid” means a compound of formula H₂NCHR^{α}COOH and “α-substituent” means the group R^{α} of said α-amino acid.

Any functional groups on the α-substituent of a natural or non-natural α-amino acid (i.e., on the groups R⁴, R⁷ and R⁸) may be protected. It will be known to the person skilled in the art that the term “protected” when used in relation to a functional substituent in a side chain of an α-amino acid means a derivative of such a substituent which is substantially non-functional. For example, carboxy groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or a NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

In a preferred embodiment R⁴, R⁷ and R⁸ are the same or different and each represents:
(i) a hydrogen atom;
(ii) a C₁₋₆ alkyl group;
(iii) a group -L³-B, in which L³ represents a bond or a C₁₋₆ alkylene group and B represents a C₆₋₁₀ aryl or 5- to 10-membered heteroaryl group;
(iv) a group of formula —CR^{p}R^{q}R^{r} in which:
   (a) R^{p}, R^{q} and R^{r} are the same or different and represent
      a hydrogen atom,
      an —OH, —SH, halogen, —CN, —CO₂H, CONH₂ or (C₁₋₄ perfluoroalkyl group, or
      a C₁₋₆ alkyl, C₂₋₆ alkenyl, C₂₋₆ alkynyl, phenyl, 5- to 6-membered heteroaryl, phenyl(C₁₋₆)alkyl, C₃₋₈ cycloalkyl, —O(C₁₋₆)alkyl, —O(C₂₋₆)alkenyl, —S(C₁₋₆)alkyl, —SO(C₁₋₆)alkyl, —SO₂(C₁₋₆) alkyl, —S(C₂₋₆)alkenyl, —SO(C₂₋₆)alkenyl or —SO₂(C₂₋₆)alkenyl group, which group is optionally substituted by a hydroxyl, —O(C₁₋₆)alkyl, phenyloxy, benzyloxy, —SH, —S(C₁₋₆)alkyl, phenylthio, benzylthio, —COOH, —CONH₂, —NHC(NH)NH₂ or

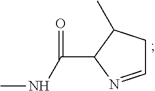

(b) two of R^{p}, R^{q} and R^{r} represent a group mentioned in (a) above and the other of R^{p}, R^{q} and R^{r} represents a group -Q-W wherein Q represents a bond or —O—, —S—, —SO— or —SO₂— and W represents a phenyl, phenyl(C₁₋₆)alkyl, C₃₋₈ carbocyclyl, C₃₋₈ cycloalkyl(C₁₋₆)alkyl, C₄₋₈ cycloalkenyl, C₄₋₈ cycloalkenyl(C₁₋₆)alkyl, 5- or 6-membered heteroaryl or 5- or 6-membered heteroaryl(C₁₋₆)alkyl group, all optionally fused to a further phenyl, 5- to 6-membered heteroaryl or 5- to 6-membered heterocyclyl ring, which group W is unsubstituted or substituted by one or more substituents which are the same or different and represent hydroxyl, halogen, —CN, —CONH₂, —CONH(C₁₋₆)alkyl, —CONH(C₁₋₆alkyl)₂, —CHO, —CH₂OH, (C₁₋₄)perfluoroalkyl, —O(C₁₋₆)alkyl, —S(C₁₋₆)alkyl, —SO(C₁₋₆)alkyl, —SO₂(C₁₋₆)alkyl, —NO₂, —NH₂, —NH(C₁₋₆)alkyl, —N((C₁₋₆)alkyl)₂, —NHCO(C₁₋₆)alkyl, (C₁₋₆)alkyl, (C₂₋₆)alkenyl, (C₂₋₆)alkynyl, (C₃₋₈)cycloalkyl, (C₄₋₈)cycloalkenyl, phenyl or benzyl; or
(c) one of R^{p}, R^{q} and R^{r} represents a group mentioned in (a) above and the other two of R^{p}, R^{q} and R^{r}, together with the carbon atom to which they are attached, form a 3 to 8-membered carbocyclyl, 5- to 6-membered heteroaryl or 5- to 6-membered heterocyclyl ring, or R^{p}, R^{q} and R^{r}, together with the carbon atom to which they are attached, form a tricyclic system; with the proviso that the group of formula —CR^{p}R^{q}R^{r} is different from the groups defined in (i), (ii) and (iii);
   or R⁷ and R⁸, taken together with the carbon to which they are attached, form a 3- to 6-membered saturated Spiro cycloalkyl or heterocyclyl ring.

In this preferred embodiment, the group of formula —CR^{p}R^{q}R^{r} is more preferably a group of formula —CR^{p}R^{q}R^{r} in which:
(a) R^{p}, R^{q} and R^{r} are the same or different and represent
   a hydrogen atom,
   an —OH, —SH, —CO₂H or CONH₂ group, or
   a C₁₋₆ alkyl, phenyl, 5- to 6-membered heteroaryl, phenyl(C₁₋₆)alkyl, C₃₋₈ cycloalkyl, —O(C₁₋₆)alkyl or —S(C₁₋₆)alkyl group, which group is optionally substituted by a hydroxyl, —O(C₁₋₆)alkyl, phenyloxy, benzyloxy, —SH, —S(C₁₋₆)alkyl, phenylthio, benzylthio, —COOH, —CONH₂ or —NHC(NH)NH₂; or
(b) two of R^{p}, R^{q} and R^{r} represent a group mentioned in (a) above and the other of R^{p}, R^{q} and R^{r} represents a group —W wherein W represents a phenyl, phenyl(C₁₋₆)alkyl, C₃₋₈ carbocyclyl, C₃₋₈ cycloalkyl(C₁₋₆)alkyl, 5- or 6-membered heteroaryl or 5- or 6-membered heteroaryl(C₁₋₆)alkyl group, all optionally fused to a further phenyl, 5- to 6-membered heteroaryl or 5- to 6-membered heterocyclyl ring, which group W is unsubstituted or substituted by a hydroxyl, halogen, —O(C₁₋₆)alkyl, —NH₂, —NH(C₁₋₆)alkyl, —N((C₁₋₆)alkyl)₂ or (C₁₋₆)alkyl.

In a more preferred embodiment of the invention, R⁴, R⁷ and R⁸ are the same or different and each represents:
(i) a hydrogen atom;
(ii) a C₁₋₆ alkyl group;
(iii) a group -L³-B, in which L³ represents a bond or a C₁₋₆ alkylene group and B represents a C₆₋₁₀ aryl or 5- to 10-membered heteroaryl group; or
(iv) a group selected from indol-3-ylmethyl, —CH₂COOH, —CH₂CH₂COOH, —CH₂CONH₂, —CH₂CH₂CONH₂, —CH₂CH₂CH₂NHC(NH)NH₂, cyclohexyl, cyclohexylmethyl and 1-benzylthio-1-methylethyl.

In one exemplary embodiment of this more preferred embodiment R⁴, R⁷ and R⁸ are the same or different and are selected from the α-substituents of the natural proteinogenic α-amino acids or an α-amino acid selected from cyclohexylglycine, t-butylserine, t-butylcysteine, tert-butylglycine and phenylglycine. For the avoidance of doubt, said natural proteinogenic α-amino acids are selected from Alanine, Arginine, Asparagine, Aspartic acid, Cysteine, Glutamic acid, Glutamine, Glycine, Histidine, Isoleucine, Leucine, Lysine, Methionine, Phenylalanine, Serine, Threonine, Tryptophan, Tyrosine and Valine. Thus, in this exemplary embodiment R⁴, R⁷ and R⁸ are the same or different and are selected from methyl, —CH₂CH₂CH₂NHC(NH)NH₂, —CH₂CONH₂, —CH₂COOH, —CH₂SH, —CH₂CH₂COOH, —CH₂CH₂CONH₂, hydrogen, imidazol-5-ylmethyl, sec-butyl, iso-butyl, —CH₂CH₂CH₂CH₂NH₃, —CH₂CH₂SCH₃, benzyl, hydroxymethyl, —CHOHCH₃, indol-3-ylmethyl, 4-hydroxyphenylmethyl, prop-2-yl, cyclohexyl, t-butoxymethyl, —CH₂SC(CH₃)₃, t-butyl and phenyl. Of these groups, presently preferred groups include hydrogen, phenyl, benzyl, iso-butyl, cyclohexyl and t-butoxymethyl.

In a still more preferred embodiment of the invention, R⁴, R⁷ and R⁸ are the same or different and each represents:
(i) a hydrogen atom;
(ii) a C₁₋₆ alkyl group; or
(iii) a group -L³-B, in which L³ represents a bond or a C₁₋₄ alkylene group and B represents a phenyl or 5- to 10-membered heteroaryl group

When any group R⁴, R⁷ or R⁸ present in group R is a

C₁₋₆ alkyl group preferably it is a C₁₋₄ alkyl group, more preferably a C₁₋₂ alkyl group, most preferably a methyl group.

When any group R⁴, R⁷ or R⁸ present in group R represents a group of formula -L³-B, preferably L³ is a bond or a C₁₋₄ alkylene group, more preferably a C₁₋₂ alkylene group, most preferably a methylene group. When any group R⁴, R⁷ or R⁸ present in group R represents a group of formula -L³-B, preferably B represents a phenyl group or a 5- to 10-membered heteroaryl group. When B represents a 5- to 10-membered heteroaryl group preferred heteroaryl groups include imidazolyl and indolyl. When any group R⁴, R⁷ or R⁸ present in group R represents a group of formula -L³-B, preferably B represents a phenyl group.

When any group R⁴, R⁷ or R⁸ present in group R is a C₁₋₆ alkyl group it is preferably unsubstituted or substituted with 1 or 2, preferably 1, unsubstituted substituents selected from halogen, C₁₋₂ alkoxy, C₁₋₂ haloalkyl, hydroxyl, —COOR′, —COONR′R″, —SR′ and —NR′R″ wherein R′ and R″ are the same or different and represent hydrogen or C₁₋₂ alkyl. When any group R⁴, R⁷ or R⁸ present in group R is a C₁₋₆ alkyl group most preferably it is unsubstituted.

When any group R⁴, R⁷ or R⁸ present in group R represents a group of formula -L³-B wherein L³ represents a C₁₋₆ alkylene group, said C₁₋₆ alkylene group is preferably unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl. More preferably said C₁₋₆ alkylene group is unsubstituted.

When any group R⁴, R⁷ or R⁸ present in group R represents a group of formula -L³-B, preferably B is unsubstituted or substituted with 1, 2 or 3, more preferably with 1 or 2, substituents which are the same or different and are selected from halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups where R′ and R″ are the same or different and represent hydrogen or unsubstituted C₁₋₂ alkyl. More preferably when any group R⁴, R⁷ or R⁸ present in group R represents a group of formula -L³-B, B is unsubstituted or substituted with one substituent selected from a halogen atom or a C₁₋₄ alkyl, C₁₋₂ alkoxy, C₁₋₂ alkylthio or hydroxy group. Most preferably B is unsubstituted. In yet more preferred embodiment R⁴, R⁷ and R⁸ are the same or different and each represents a hydrogen atom, an unsubstituted C₁₋₆ alkyl group or a group -L³-B where L³ represents a bond or an unsubstituted C₁₋₂ alkylene group and B represents an unsubstituted phenyl group or a phenyl group substituted with one substituent selected from a halogen atom or a C₁₋₂ alkyl, C₁₋₂ alkoxy, C₁₋₂ alkylthio or hydroxy group.

Particularly preferred R⁴, R⁷ and R⁸ groups are hydrogen atoms and unsubstituted C₁₋₆ alkyl groups. Most preferred R⁴, R⁷ and R⁸ groups include hydrogen atoms and unsubstituted methyl, ethyl, n-propyl, i-propyl, n-butyl, i-butyl, sec-butyl and t-butyl groups.

Preferably when R is a group of formula (X1) at least one of the substituents R⁷ and R⁸ is a C₁₋₆ alkyl group, for example methyl, ethyl, n- or iso-propyl or n-, iso-, sec- or t-butyl.

Preferably in (X1) at least one of R⁷ and R⁸ is a hydrogen atom.

Most preferably when R is a group of formula (X1) R⁷ and R⁸ are the same or different and represent a hydrogen atom or an unsubstituted C₁₋₆ alkyl, phenyl, 5- to 6-membered heteroaryl, C₃₋₈ carbocyclyl, C₃₋₈ cycloalkyl(C₁₋₆)alkyl, or phenyl(C₁₋₆)alkyl group. Where one of R⁷ or R⁸ is hydrogen, then preferably the other of R⁷ and R⁸ is other than hydrogen. More preferably R⁷ and R⁸ are the same or different and represent hydrogen or unsubstituted C₁₋₆ alkyl. Where neither R⁷ nor R⁸ are hydrogen, preferably R⁷ and R⁸ are the same or different and represent unsubstituted C₁₋₂ alkyl groups. More preferably when neither R⁷ nor R⁸ are hydrogen, R⁷ and R⁸ are both unsubstituted methyl groups.

When R represents either (i) a group of formula (X1), where one of R⁷ and R⁸ is hydrogen and the other of R⁷ and R⁸ is a group other than hydrogen, or (ii) a group of formula (Y1), where R⁷ is hydrogen, then preferably the group R has L-isomerism. For the avoidance of doubt, the group R has L-isomerism when the carbon atom directly bound to the group R² represents a chiral centre having L-isomerism that corresponds to the L-isomerism possessed by the natural proteinogenic amino acids about their α-carbon atom. In this embodiment, therefore, the stereochemistry is as illustrated below:

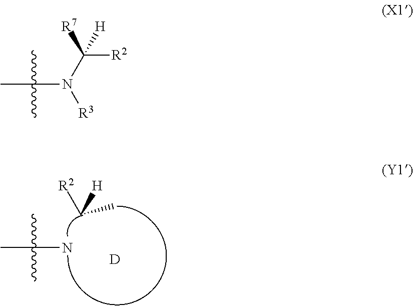

Preferably R is a group of formula (X1) or (Y1). Most preferably R is a group of formula (X1). Compounds where R is a group of formula (X1) or (Y1) are particularly well suited to systemic administration regimes.

In a preferred embodiment the group R is a group of formula (X1) or (Y1) wherein the nitrogen moiety drawn out explicitly in that group is not directly linked to a carbonyl (—C(═O)—). More preferably the group R is a group of formula (X1) wherein the nitrogen moiety drawn out explicitly in that group is not directly linked to a carbonyl (—C(═O)—).

In an alternative embodiment, group R is a group of formula (X2) or (Y2). Such compounds are particularly well suited to local, or topical, administration regimes.

In a preferred embodiment where A¹ represents an unfused 1,3-phenylene group any group R⁴, R⁷ or R⁸ that is present in the group R is a group other than hydrogen. In a particularly preferred embodiment where A¹ represents an unfused 1,3-phenylene group any group R⁴, R⁷ or R⁸ that is present in the group R is the same or different and represents an unsubstituted C₁₋₆ alkyl group. In a most preferred embodiment here A¹ represents an unfused 1,3-phenylene group, R is a group of formula (X1) and R⁷ and R⁸ are the same or different and represent an unsubstituted C₁₋₆ alkyl group.

R² is either a carboxylic acid group —COOH or an ester group —COOR⁹. The term “ester” or “esterified carboxyl group” in connection with substituent R² above means a group —(C═O)OR⁹ in which R⁹ is the group characterising the ester, notionally derived from the alcohol R⁹—OH. In one embodiment, R² is preferably an ester group —COOR⁹, i.e. it is preferably an ester group which is hydrolysable by one or more cellular carboxylesterase enzymes to a —COOH group.

Where R² is an ester group, it must be one which in the compound of the invention is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes other enzymes such as biphenylhydrolase (BPH) may also have a role in hydrolysing the conjugates. In general, if the carboxylesterase hydrolyses the free amino acid ester to the parent acid it will also hydrolyse the ester motif when covalently conjugated to the IKK inhibitor. Hence, the broken cell assay described later provides a straightforward, quick and simple first screen for esters which have the required hydrolysis profile. Ester motifs selected in that way may then be re-assayed in the same carboxylesterase assay when conjugated to the IKK inhibitor via the chosen conjugation chemistry, to confirm that it is still a carboxylesterase substrate in that background.

Subject to the requirement that they be hydrolysable by intracellular carboxylesterase enzymes, examples of particular ester groups R² include those of formula —(C═O)OR⁹ wherein R⁹ is —CR¹⁴R¹⁵R¹⁶ wherein:
(i) R¹⁵ represents hydrogen or a group of formula —[C₁₋₄ alkylene]_{b}-(Z¹)ₐ—[C₁₋₄ alkyl] or —[C₁₋₄ alkylene]_{b}-(Z¹)ₐ—[C₂₋₄ alkenyl] wherein a and b are the same or different and represent 0 or 1, and Z¹ represents —O—, —S—, or —NR¹⁷— wherein R¹⁷ is hydrogen or C₁₋₄ alkyl, R¹⁶ represents hydrogen or C₁₋₄ alkyl, and R¹⁴ represents hydrogen or C₁₋₄ alkyl;
(ii) R¹⁵ represents a phenyl or a 5- to 10-membered heteroaryl group optionally fused to a further phenyl, 5- to 10-membered heteroaryl, C₃₋₇ carbocyclyl or 5- to 10-membered heterocyclyl group, R¹⁶ represents hydrogen or C₁₋₄ alkyl, and R¹⁴ represents hydrogen;

(iii) R¹⁵ represents a group of formula -(Alk⁵)—NR¹⁸R¹⁹ wherein Alk⁵ represents a C₁₋₄ alkylene group and either (a) R¹⁸ and R¹⁹ are the same or different and represent hydrogen or C₁₋₄ alkyl, or (b) R¹⁸ and R¹⁹, together with the nitrogen atom to which they are bonded, form a 5- to 10-membered heteroaryl or 5- to 10-membered heterocyclyl group optionally fused to a further phenyl, 5- to 10-membered heteroaryl, C₃₋₇ carbocyclyl or 5- to 10-membered heterocyclyl group; R¹⁶ represents hydrogen or C₁₋₄ alkyl, and R¹⁴ represents hydrogen; or
(iv) R¹⁵ and R¹⁶, together with the carbon atom to which they are bonded, form a phenyl, 5- to 10-membered heteroaryl, C₃₋₇ carbocyclyl or 5- to 10-membered heterocyclyl group which is optionally fused to a further phenyl, 5- to 10-membered heteroaryl, C₃₋₇ carbocyclyl or 5- to 10-membered heterocyclyl group, and R¹⁴ represents hydrogen.

Preferred substituents on the alkyl, alkylene and alkenyl groups in R¹⁴, R¹⁵, R¹⁶, R¹⁷, R¹⁸, R¹⁹ and Alk⁵ groups include one or two substituents which are the same or different and are selected from halogen, C₁₋₄ alkyl, C₂₋₄ alkenyl, C₁₋₄ alkoxy, hydroxyl and —NR′R″ wherein and R″ are the same or different and represent hydrogen or C₁₋₂ alkyl. More preferred substituents are halogen, C₁₋₂ alkoxy, hydroxyl and —NR′R″wherein R′ and R″ are the same or different and represent hydrogen or C₁₋₂ alkyl. Most preferably the alkyl, alkylene and alkenyl groups in R¹⁵, R¹⁶ and Alk⁵ are unsubstituted.

Preferred substituents on the phenyl, heteroaryl, carbocyclyl and heterocyclyl groups in or formed by R¹⁵, R¹⁶, R¹⁸ and R¹⁹ groups include one or two substituents which are the same or different and are selected from halogen atoms and C₁₋₄ alkyl, C₁₋₄ alkylene, C₁₋₄ alkoxy, C₁₋₄ haloalkyl, hydroxyl, cyano, nitro and —NR′R″ groups wherein each R′ and R″ is the same or different and represents hydrogen or C₁₋₄ alkyl, more preferably halogen atoms and C₁₋₂ alkyl, C₁₋₂ alkylene, C₁₋₂ alkoxy and hydroxyl groups. More preferably the phenyl, heteroaryl, carbocyclyl and heterocyclyl groups in - or formed by R¹⁵, R¹⁶, R¹⁸ and R¹⁹ are unsubstituted or substituted by a C₁₋₂ alkylene group, in particular a methylene group. Most preferably the phenyl, heteroaryl, carbocyclyl and heterocyclyl groups in or formed by R¹⁵, R¹⁶, R¹⁸ and R¹⁹ are unsubstituted.

When R¹⁵ represents a group of formula —[C₁₋₄ alkylene]_{b}-(Z¹)ₐ—[C₁₋₄ alkyl], preferably either a or b is zero, for example both a and b are zero. When [C₁₋₄ alkylene] is present, it is preferably a C₁₋₃ alkylene, more preferably a C₁₋₂ alkylene such as a group —CH₂—CH₂—.

When R¹⁵ represents a group of formula —[C₁₋₄ alkylene]_{b}-(Z¹)ₐ—[C₁₋₄ alkyl], preferably C₁₋₄ alkyl is a C₁₋₃ alkyl group such as methyl, ethyl or n-propyl, most preferably methyl.

When R¹⁵ represents a group of formula —[C₁₋₄ alkylene]_{b}-(Z¹)ₐ—[C₁₋₄ alkyl] and a is 1, Z¹ is preferably —O— or —NR¹⁷— wherein R¹⁷ is hydrogen or C₁₋₂ alkyl, more preferably Z′ is —O—.

When R¹⁵ represents a group of formula —[C₁₋₄ alkylene]_{b}-(Z¹)ₐ—[C₂₋₄ alkenyl], preferably either a or b is zero, more preferably both a and b are zero. When [C₁₋₄ alkylene] is present, it is preferably a C₁₋₃ alkylene, more preferably a C₁₋₂ alkylene.

When R¹⁵ represents a group of formula —[C₁₋₄ alkylene]_{b}-(Z¹)ₐ—[C₂₋₄ alkenyl], preferably C₂₋₄ alkenyl is a C₂₋₃ alkenyl group, in particular —CH═CH₂.

When R¹⁵ represents a group of formula —[C₁₋₄ alkylene]_{b}-(Z¹)ₐ—[C₁₋₄ alkenyl] and a is 1, Z′ is preferably —O— or —NR¹⁷— wherein R¹⁷ is hydrogen or C₁₋₂ alkyl, more preferably Z¹ is —O—. Most preferably Z¹ is absent (i.e. a is zero).

When R¹⁵ represents hydrogen or a group of formula —[C₁₋₄ alkylene]_{b}-(Z¹)ₐ—[C₁₋₄ alkyl] or —[C₁₋₄ alkylene]_{b}-(Z¹)ₐ—[C₂₋₄ alkenyl], preferably R¹⁵ represents hydrogen or a C₁₋₄ alkyl or C₂₋₄ alkenyl group, or a group —(C₁₋₄ alkyl)-O—(C₁₋₄ alkyl). More preferably

R¹⁵ represents hydrogen, methyl, ethyl, n-propyl, —CH═CH₂ or —CH₂—CH₂—O—CH₃, most preferably methyl.

When R¹⁵ represents hydrogen or a group of formula —[C₁₋₄ alkylene]_{b}-(Z¹)ₐ—[C₁₋₄ alkyl] or —[C₁₋₄ alkylene]_{b}-(Z¹)ₐ—[C₂₋₄ alkenyl], preferably R¹⁶ represents hydrogen or C₁₋₂ alkyl, more preferably hydrogen or methyl.

When R¹⁵ represents hydrogen or a group of formula —[C₁₋₄ alkylene]_{b}-(Z¹)_{c}—[C₁₋₄ alkyl] or —[C₁₋₄ alkylene]_{b}-(Z¹)ₐ—[C₂₋₄ alkenyl], preferably R¹⁴ represents hydrogen or C₁₋₂ alkyl, more preferably R¹⁴ represents hydrogen or methyl.

When R¹⁵ represents hydrogen or a group of formula —[C₁₋₄ alkylene]_{b}-(Z¹)ₐ—[C₁₋₄ alkyl] or —[C₁₋₄ alkylene]_{b}-(Z¹)ₐ—[C₂₋₄ alkenyl], preferably the alkyl, alkylene and alkenyl groups in both R¹⁵ and R¹⁶ are unsubstituted.

When R¹⁵ represents a phenyl or a 5- to 10-membered heteroaryl group optionally fused to a further phenyl, 5- to 10-membered heteroaryl, C₃₋₇ carbocyclyl or 5- to 10-membered heterocyclyl group, preferably it represents a non-fused phenyl or a non-fused 5- to 6-membered heteroaryl group. Preferred heteroaryl groups include pyridyl, pyrrolyl, isothiazolyl, pyrazolyl and isoxazolyl, most preferably pyridyl.

When R¹⁵ represents a phenyl or a 5- to 10-membered heteroaryl group optionally fused to a further phenyl, 5- to 10-membered heteroaryl, C₃₋₇ carbocyclyl or 5- to 10-membered heterocyclyl group, preferably the phenyl, heteroaryl, carbocyclyl and heterocyclyl groups in R⁹ are unsubstituted.

When R¹⁵ represents a phenyl or a 5- to 10-membered heteroaryl group optionally fused to a further phenyl, 5- to 10-membered heteroaryl, C₃₋₇ carbocyclyl or 5- to 10-membered heterocyclyl group, R¹⁶ preferably represents hydrogen or C₁₋₄ alkyl, more preferably hydrogen or C₁₋₂ alkyl, most preferably hydrogen. Preferably the C₁₋₄ alkyl groups of R¹⁶ are unsubstituted.

When R¹⁵ represents a group of formula -(Alk⁵)—NR¹⁸R¹⁹, Alk⁵ preferably represents a C₁₋₂ alkylene group, preferably either —CH₂— or —CH₂CH₂—.

When R¹⁵ represents a group of formula -(Alk⁵)—NR¹⁸R¹⁹ and R¹⁸ and R¹⁹ are the same or different and represent hydrogen or C₁₋₄ alkyl, preferably R¹⁸ represents hydrogen or C₁₋₂ alkyl, more preferably R¹⁸ represents a methyl group. When R¹⁵ represents a group of formula -(Alk⁵)—NR¹⁸R¹⁹ and R¹⁸ and R¹⁹ are the same or different and represent hydrogen or C₁₋₄ alkyl, preferably R¹⁹ represents hydrogen or C₁₋₂ alkyl, more preferably R¹⁹ represents a methyl group.

When R¹⁵ represents a group of formula -(Alk⁵)—NR¹⁸R¹⁹ and R¹⁸ and R¹⁹, together with the nitrogen atom to which they are bonded, form a 5- to 10-membered heteroaryl or 5- to 10-membered heterocyclyl group optionally fused to a further phenyl, 5- to 10-membered heteroaryl, C₃₋₇ carbocyclyl or 5- to 10-membered heterocyclyl group, preferably they form a non-fused 5- to 6-membered heteroaryl or non-fused 5- to 6-membered heterocyclyl group. More preferably they form a 5- to 6-membered heterocyclyl group. Preferred heterocyclyl groups include piperidinyl, piperazinyl, morpholinyl and pyrrolidinyl, most preferably morpholinyl.

When R¹⁵ represents a group of formula -(Alk⁵)—NR¹⁸R¹⁹, Alk⁵ preferably represents a C₁₋₂ alkylene group, more preferably a group —CH₂CH₂—.

When R¹⁵ represents a group of formula -(Alk⁵)—NR¹⁸R¹⁹, R¹⁶ preferably represents hydrogen or C₁₋₂ alkyl, most preferably hydrogen.

When R¹⁵ represents a group of formula -(Alk⁵)—NR¹⁸R¹⁹, preferably the alkyl and alkylene groups in Alk⁵, R¹⁸ and R¹⁹ are unsubstituted. When R¹⁵ represents a group of formula -(Alk⁵)—NR¹⁸R¹⁹, preferably the phenyl, heteroaryl, carbocyclyl and heterocyclyl groups in R¹⁸ and R¹⁹ are unsubstituted.

When R¹⁵ represents a group of formula -(Alk⁵)—NR¹⁸R¹⁹, preferred groups include —CH₂—CH₂—NMe₂ and —CH₂—CH₂-morpholinyl.

When R¹⁵ and R¹⁶, together with the carbon atom to which they are bonded, form a phenyl, 5- to 10-membered heteroaryl, C₃₋₇ carbocyclyl or 5- to 10-membered heterocyclyl group which is optionally fused to a further phenyl, 5- to 10-membered heteroaryl, C₃₋₇ carbocyclyl or 5- to 10-membered heterocyclyl group, preferred groups include non-fused phenyl, non-fused 5- to 6-membered heteroaryl, non-fused 5- to 6-membered heterocyclyl, non-fused C₃₋₇ carbocyclyl and C₃₋₇ carbocyclyl fused to a phenyl ring, more preferably non-fused phenyl, non-fused 5- to 6-membered heterocyclyl, non-fused C₃₋₇ carbocyclyl and C₃₋₇ carbocyclyl fused to a phenyl ring.

When R¹⁵ and R¹⁶ form a cyclic group together with the carbon atom to which they are bonded, preferred non-fused 5- to 6-membered heterocyclyl groups include piperidinyl, tetrahydrofuranyl, piperazinyl, morpholinyl and pyrrolidinyl groups, more preferably piperidinyl and tetrahydrofuranyl groups. When R¹⁵ and R¹⁶ form a cyclic group together with the carbon atom to which they are bonded, preferred non-fused C₃₋₇ carbocyclyl groups include cyclopentyl and cyclohexyl, more preferably cyclopentyl. When R¹⁵ and R¹⁶ form a cyclic group together with the carbon atom to which they are bonded, preferred C₃₋₇ carbocyclyl groups fused to a phenyl ring include indanyl.

When R¹⁵ and R¹⁶ form a cyclic group together with the carbon atom to which they are bonded, preferably the phenyl, heteroaryl, carbocyclyl and heterocyclyl groups formed are unsubstituted or substituted by one or two substituents which are the same or different and are selected from halogen atoms and C₁₋₄ alkyl, C₁₋₄ alkylene, C₁₋₄ alkoxy, C₁₋₄ haloalkyl, hydroxyl, cyano, nitro and —NR′R″ groups wherein each R′ and R″ is the same or different and represents hydrogen or C₁₋₄ alkyl, more preferably selected from halogen atoms or C₁₋₂ alkyl, C₁₋₂ alkylene, C₁₋₂ alkoxy and hydroxyl groups. Most preferably the phenyl, heteroaryl, carbocyclyl and heterocyclyl groups formed are unsubstituted or substituted by a C₁₋₂ alkyl group (such as a methyl group) or by a C₁₋₂ alkylene group (such as by a methylene group). Even more preferably the phenyl, heteroaryl, carbocyclyl and heterocyclyl groups so formed are unsubstituted.

Preferred R² groups are —COOH and —COOR^{S} where R⁹ represents C₁₋₄ alkyl groups (such as methyl, ethyl, n- or iso-propyl and n-, sec- and tert-butyl), C₃₋₇ carbocyclyl groups (such as cyclopentyl and cyclohexyl), C₂₋₄ alkenyl groups (such as allyl), and also phenyl, benzyl, 2-pyridylmethyl, 3-pyridylmethyl, 4-pyridylmethyl, N-methylpiperidin-4-yl, tetrahydrofuran-3-yl, methoxyethyl, indanyl, norbonyl, dimethylaminoethyl and morpholinoethyl groups. More preferably R⁹ represents C₁₋₄ alkyl or C₃₋₇ carbocyclyl.

Preferably said preferred R⁹ groups are unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R¹ and R″ are the same or different and represent hydrogen or unsubstituted C₁₋₂ alkyl. More preferably said preferred R⁹ groups are unsubstituted or substituted with 1 or 2 unsubstituted substituents which are the same or different and are selected from halogen atoms and C₁₋₂ alkoxy and C₁₋₂ haloalkyl groups. Most preferably said preferred R⁹ groups are unsubstituted. When said preferred R⁹ groups are substituted, preferably at most one substituent is a hydroxyl group.

When R² is —COOR⁹ more preferably R⁹ represents unsubstituted C₁₋₄ alkyl, unsubstituted C₃₋₇ carbocyclyl or unsubstituted C₂₋₄ alkenyl. More preferably R⁹ is cyclopentyl or t-butyl; most preferably R⁹ is cyclopentyl.

Compounds where R² represents —COOH or —COOR⁹ wherein R⁹ is C₁₋₄ alkyl or C₃₋₇ carbocyclyl can be described by a group where R² is —COOR¹⁰ and R¹⁰ is hydrogen, C₁₋₄ alkyl or C₃₋₇ carbocyclyl. Preferably R² is —COOR¹⁰ where R¹⁰ is hydrogen or C₃₋₇ carbocyclyl, more preferably where R¹⁰ is hydrogen or cyclopentyl. In one embodiment, R¹⁰ is other than hydrogen, i.e. is selected from C₁₋₄ alkyl or C₃₋₇ carbocyclyl as described above.

Macrophages are known to play a key role in inflammatory disorders through the release of cytokines in particular TNF-α and IL-1. In rheumatoid arthritis they are major contributors to the maintenance of joint inflammation and joint destruction. Macrophages are also involved in tumour growth and development. Hence agents that selectively target macrophage cell proliferation could be of value in the treatment of cancer and autoimmune disease. Targeting specific cell types would be expected to lead to reduced side-effects. The inventors have discovered a method of targeting inhibitors to macrophages and other cells derived from the myelo-monocytic lineage such as monocytes, osteoclasts and dendritic cells. This is based on the observation that the way in which the esterase motif is linked to the inhibitor determines whether it is hydrolysed, and hence whether or not it accumulates in different cell types. Specifically it has been found that macrophages and other cells derived from the myelo-monocytic lineage contain the human carboxylesterase hCE-1 whereas other cell types do not. In the general formulae (IA) and (IB) when the group R is a group of formula (X1) or (Y1) whose nitrogen moiety is not directly linked to a carbonyl (—C(═O)—), the ester will only be hydrolysed by hCE-1 and hence the inhibitors will selectively accumulate in macrophage-related cells. For the avoidance of doubt, the nitrogen moiety referred to here is the N atom drawn out explicitly in the groups (X1) and (Y1). Herein, unless “monocyte” or “monocytes” is specified, the term macrophage or macrophages will be used to denote macrophages (including tumour associated macrophages) and/or monocytes.

In a preferred embodiment (1) of the invention there is provided a compound of the present invention in which:
L¹ represents C₁₋₄ alkylene which is unsubstituted or substituted with 1 or 2 unsubstituted substituents which are the same or different and are selected from halogen atoms and C₁₋₂ alkoxy and C₁₋₂ haloalkyl groups;
A¹ represents an unfused phenyl or a 5- to 6-membered heteroaryl group which is unsubstituted or substituted by 1, 2 or 3 substituents which are the same or different and are selected from halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups wherein R′ and R″ are the same or different and represent hydrogen or unsubstituted C₁₋₂ alkyl;
Alk² represents an unsubstituted C₁₋₃ alkylene, C₂₋₃ alkenylene or C₂₋₃ alkynylene group;
Alk³ represents an unsubstituted C₁₋₄ alkylene group;
A² represents an unfused phenyl which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl;
Het represents —O—, —NH— or —S—;
Alk¹ represents a bond or a C₁₋₄ alkylene group which is unsubstituted or substituted with 1 or 2 unsubstituted substituents selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl, or Alk¹ represents a group -A³-Alk⁴- where A³ represents an unfused phenyl or unfused 5- to 6-membered heteroaryl group which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl, and Alk⁴ represents an unsubstituted C₁₋₃ alkylene group;
R represents a group of formula (X1) or (Y1):

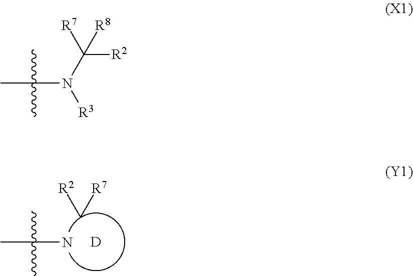

in which
R² represents —COOH or —COOR⁹ wherein R⁹ represents a C₁₋₄ alkyl, C₃₋₇ carbocyclyl or C₂₋₄ alkenyl group, or R⁹ represents a phenyl, benzyl, 2-pyridylmethyl, 3-pyridylmethyl, 4-pyridylmethyl, N-methylpiperidin-4-yl, tetrahydrofuran-3-yl, methoxyethyl, indanyl, norbonyl, dimethylaminoethyl or morpholinoethyl group, said R⁹ being unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl;
R³ represents a hydrogen atom or an unsubstituted methyl group;
R⁷ and R⁸, which are the same or different, represent a hydrogen atom or an unsubstituted C₁₋₆ alkyl group; and
Ring D represents an unfused unsubstituted 5- to 6-membered heterocyclyl group.

In a more preferred embodiment (2), the present invention provides a compound which is (a) a thiophene carboxamide derivative of formula (IA′) or (IB′), or a tautomer thereof; or (b) a pharmaceutically acceptable salt, N-oxide, hydrate or solvate thereof:

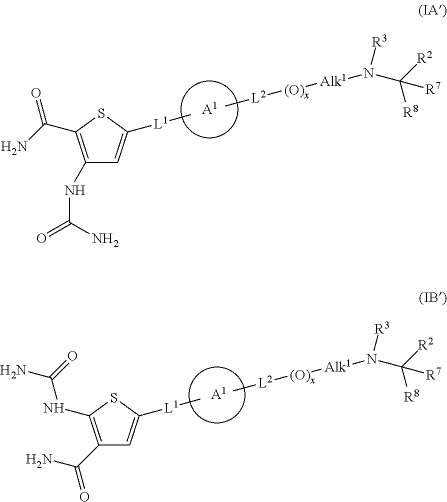

wherein:
L¹ represents unsubstituted C₁₋₄ alkylene;
A¹ represents 1,4-phenylene or 1,3-phenylene, which is unsubstituted or substituted by 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl;
L² represents -Alk²-, -Alk²-A²- or -Alk²-Alk³-;
Alk² represents an unsubstituted C₁₋₃ alkylene, C₂₋₃ alkenylene or C₂₋₃ alkynylene group;
Alk³ represents an unsubstituted C₁₋₄ alkylene group;
A² represents an unfused phenyl which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl;
x is 0 or 1;
Alk¹ represents a bond or a C₁₋₄ alkylene group which is unsubstituted or substituted with 1 or 2 unsubstituted substituents selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl;
R² represents —COOR^{S} wherein R⁹ represents an unsubstituted C₁₋₄ alkyl, C₃₋₇ carbocyclyl or C₂₋₄ alkenyl group;
R³ represents a hydrogen atom or an unsubstituted methyl group; and
R⁷ and R⁸, which are the same or different, represent a hydrogen atom or an unsubstituted C₁₋₆ alkyl group.

In a particularly preferred embodiment (3), the present invention provides a compound which is: (a) a thiophene carboxamide derivative of formula (IC) or (ID), or a tautomer thereof; or (b) a pharmaceutically acceptable salt, N-oxide, hydrate or solvate thereof:

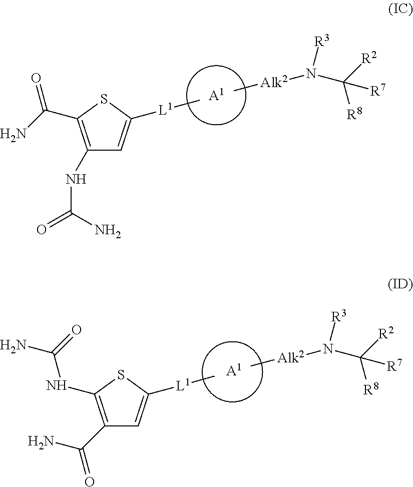

wherein:
L¹ represents unsubstituted C₁₋₄ alkylene;
A¹ represents 1,4-phenylene or 1,3-phenylene, which is unsubstituted or substituted by 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl;
Alk² represents an unsubstituted C₁₋₃ alkylene, C₂₋₃ alkenylene or C₂₋₃ alkynylene group;
R² represents —COOR⁹ wherein R⁹ represents an unsubstituted C₁₋₄ alkyl, C₃₋₇ carbocyclyl or C₂₋₄ alkenyl group;
R³ represents a hydrogen atom or an unsubstituted methyl group; and
R⁷ and R⁸, which are the same or different, represent a hydrogen atom or an unsubstituted C₁₋₆ alkyl group.

Preferred compounds of the present invention are:
Cyclopentyl 2-(4-((4-carbamoyl-5-ureidothiophen-2-yl)methyl)benzylamino)-4-methylpentanoate;
Tert-butyl 2-(4-((4-carbamoyl-5-ureidothiophen-2-yl)methyl)benzylamino)-4-methylpentanoate;
Cyclopentyl 2-(4-((4-carbamoyl-5-ureidothiophen-2-yl)methyl)benzylamino)-4-methylpentanoate;
Tert-butyl 2-(4-((4-carbamoyl-5-ureidothiophen-2-yl)methyl)benzylamino)-4-methylpentanoate;
Cyclopentyl 2-(4-((4-carbamoyl-5-ureidothiophen-2-yl)ethynyl)benzylamino)-4-methylpentanoate;
Tert-butyl 2-(4-((4-carbamoyl-5-ureidothiophen-2-yl)ethynyl)benzylamino)-4-methylpentanoate;
2-(4-((4-carbamoyl-5-ureidothiophen-2-yl)methyl)benzylamino)-4-methylpentanoic acid;
2-(4-((4-carbamoyl-5-ureidothiophen-2-yl)methyl)benzylamino)-4-methylpentanoic acid; and
2-(4-((4-carbamoyl-5-ureidothiophen-2-yl)ethynyl)benzylamino)-4-methylpentanoic acid.

Particularly preferred compounds of the invention are:
Cyclopentyl 2-(4-((4-carbamoyl-5-ureidothiophen-2-yl)methyl)benzylamino)-4-methylpentanoate;
Tert-butyl 2-(4-((4-carbamoyl-5-ureidothiophen-2-yl)methyl)benzylamino)-4-methylpentanoate;
Cyclopentyl 2-(4-((4-carbamoyl-5-ureidothiophen-2-yl)methyl)benzylamino)-4-methylpentanoate;
Tert-butyl 2-(4-((4-carbamoyl-5-ureidothiophen-2-yl)methyl)benzylamino)-4-methylpentanoate;
Cyclopentyl 2-(4-((4-carbamoyl-5-ureidothiophen-2-yl)ethynyl)benzylamino)-4-methylpentanoate; and
Tert-butyl 2-(4-((4-carbamoyl-5-ureidothiophen-2-yl)ethynyl)benzylamino)-4-methylpentanoate.

Presently most preferred compounds of the invention are:
Cyclopentyl 2-(4-((4-carbamoyl-5-ureidothiophen-2-yl)methyl)benzylamino)-4-methylpentanoate;
Tert-butyl 2-(4-((4-carbamoyl-5-ureidothiophen-2-yl)methyl)benzylamino)-4-methylpentanoate;
Cyclopentyl 2-(4-((4-carbamoyl-5-ureidothiophen-2-yl)methyl)benzylamino)-4-methylpentanoate; and
Tert-butyl 2-(4-((4-carbamoyl-5-ureidothiophen-2-yl)methyl)benzylamino)-4-methylpentanoate.

The compounds with which the invention is concerned are inhibitors of IKK, especially IKKβ kinase activity, and are therefore of use in the treatment of diseases modulated by IKK activity and the NF-κB cascade. Such diseases include neoplastic/proliferative, immune and inflammatory disease. In particular, uses of the compounds include: treatment of cancer, such as hepatocellular cancer or melanoma, but also including bowel cancer, ovarian cancer, head and neck and cervical squamous cancers, gastric or lung cancers, anaplastic oligodendrogliomas, glioblastoma multiforme or medulloblastomas; and treatment of rheumatoid arthritis, psoriasis, inflammatory bowel disease, Crohn's disease, ulcerative colitis, chronic obstructive pulmonary disease, asthma, multiple sclerosis, diabetes, such as type II diabetes mellitus, atopic dermatitis, graft versus host disease, or systemic lupus erythematosus.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, but an exemplary dosage would be 0.1-1000 mg per day.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers, for example lactose, sugar, maize-starch, calcium phosphate, sorbitol or glycine; tabletting lubricant, for example magnesium stearate, talc, polyethylene glycol or silica; disintegrants, for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavourings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds of the invention may be used in conjunction with a number of known pharmaceutically active substances. For example, the compounds of the invention may be used with cytotoxics, HDAC inhibitors, kinase inhibitors, aminopeptidase inhibitors and monoclonal antibodies (for example those directed at growth factor receptors). Preferred cytotoxics include, for example, taxanes, platins, anti-metabolites such as 5-fluoracil, topoisomerase inhibitors and the like. The medicaments of the invention comprising amino acid derivatives of formula (IA) or (IB), tautomers thereof or pharmaceutically acceptable salts, N-oxides, hydrates or solvates thereof therefore typically further comprise a cytotoxic, an HDAC inhibitor, a kinase inhibitor, an aminopeptidase inhibitor and/or a monoclonal antibody.

Further, the present invention provides a pharmaceutical composition comprising:
(a) a compound which is: (i) a thiophene carboxamide derivative of formula (IA) or (IB), or a tautomer thereof; or (ii) a pharmaceutically acceptable salt, N-oxide, hydrate or solvate thereof;
(b) a cytotoxic agent, an HDAC inhibitor, a kinase inhibitor, an aminopeptidase inhibitor and/or a monoclonal antibody; and
(c) a pharmaceutically acceptable carrier or diluent.

Also provided is a product comprising:
(a) a compound which is: (i) a thiophene carboxamide derivative of formula (IA) or (IB), or a tautomer thereof; or (ii) a pharmaceutically acceptable salt, N-oxide, hydrate or solvate thereof; and
(b) a cytotoxic agent, an HDAC inhibitor, a kinase inhibitor, an aminopeptidase inhibitor and/or a monoclonal antibody,

for the separate, simultaneous or sequential use in the treatment of the human or animal body.

### Synthesis

There are multiple synthetic strategies for the synthesis of the compounds of formula (IA) or (IB) with which the present invention is concerned, but all rely on known chemistry, known to the synthetic organic chemist. Thus, compounds according to formula (IA) or (IB) can be synthesised according to procedures described in the standard literature and well known to those skilled in the art. Typical literature sources are “Advanced organic chemistry”, 4^{th} Edition (Wiley), J March, “Comprehensive Organic Transformation”, 2^{nd} Edition (Wiley), R. C. Larock , “Handbook of Heterocyclic Chemistry”, 2^{nd} Edition (Pergamon), A. R. Katritzky, review articles such as found in “Synthesis”, “Acc. Chem. Res.”, “Chem. Rev”, or primary literature sources identified by standard literature searches online or from secondary sources such as “Chemical Abstracts” or “Beilstein”.

The compounds of the invention may be prepared by a number of processes generally described below and more specifically in the Examples hereinafter. In the reactions described below, it may be necessary to protect reactive functional groups, for example hydroxyl, amino and carboxy groups, where these are desired in the final product, to avoid their unwanted participation in the reactions [see for example Greene, T. W., “Protecting Groups in Organic Synthesis”, John Wiley and Sons, 1999]. Conventional protecting groups may be used in conjunction with standard practice. In some instances deprotection may be the final step in the synthesis of a compound of general formula (IA) or (IB), and the processes according to the invention described herein after are understood to extend to such removal of protecting groups.

The amino acid ester building blocks can be prepared in a number of ways. Scheme 1 illustrates the main routes employed for their preparation for the purpose of this application. To the chemist skilled in the art it will be apparent that there are other methodologies that will also achieve the preparation of these intermediates.

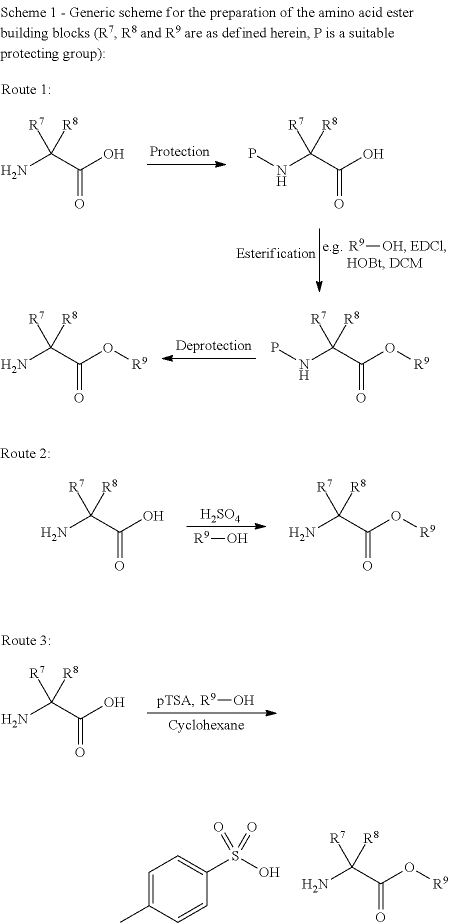

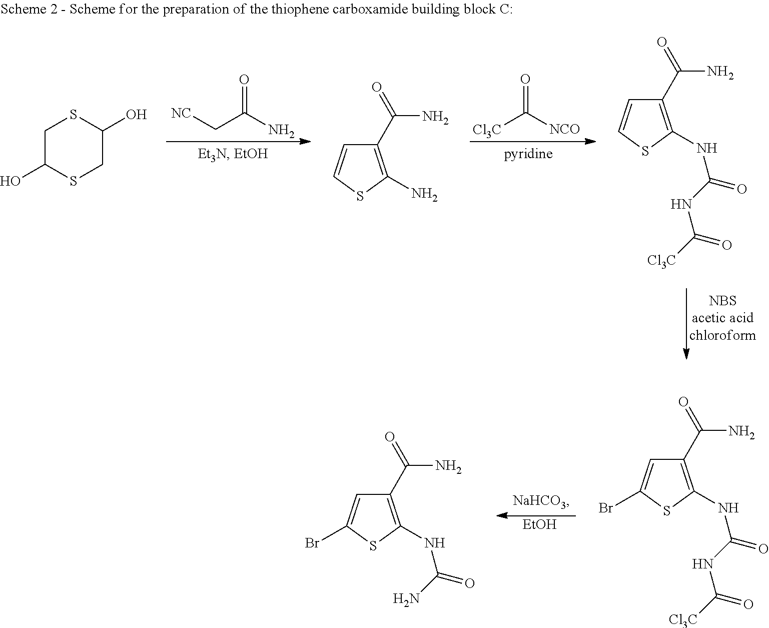

It will be apparent to the individual skilled in the art that the general route set out in scheme 2 is one of a number available. For example, methods of preparation of a suitable thiophene carboxamide core are described in detail in WO 03/104218, the content of which is herein incorporated by reference in its entirety.

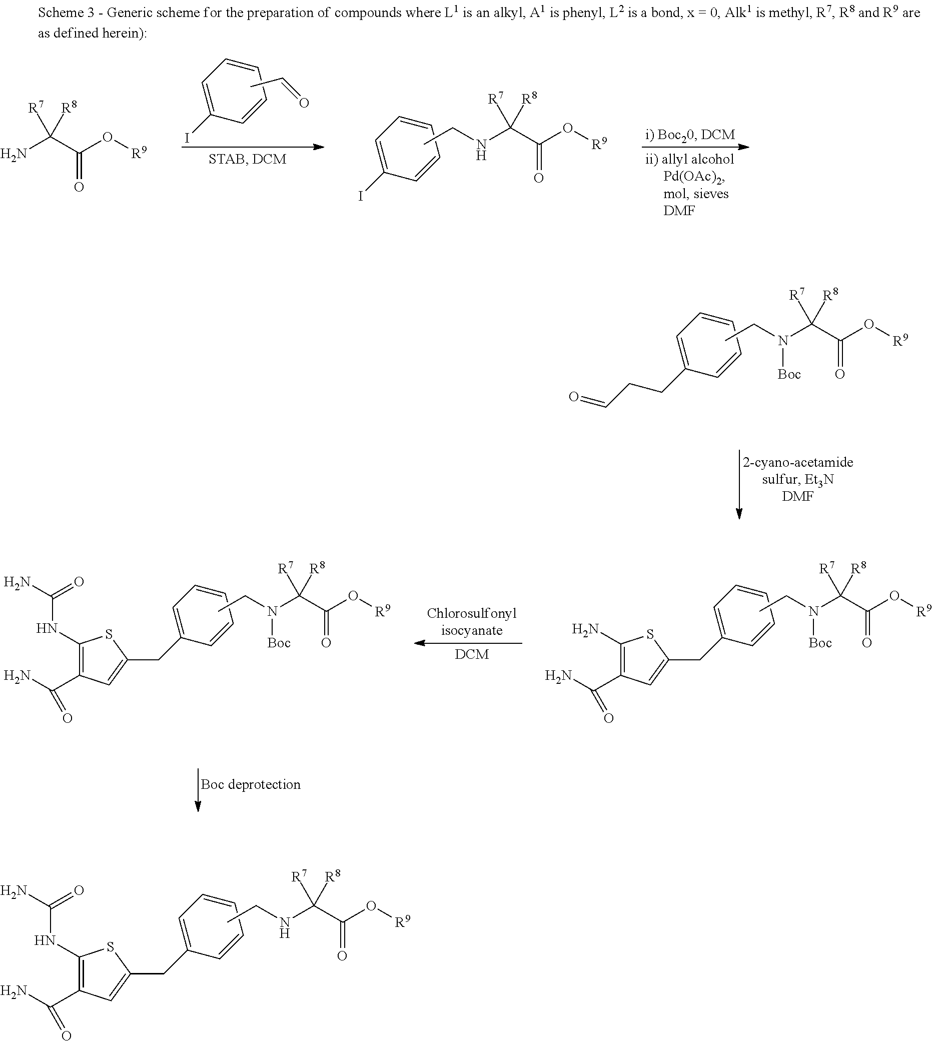

Scheme 3 shows a process that involves reductive amination of the amino acid ester with the corresponding benzaldehyde, followed by N-protection and a Heck reaction giving rise to the key aldehyde intermediate. This is transformed into the thiophene ring using 2-cyano acetamide. It will be apparent to a person skilled in art that the nature of the group L¹ will have an impact on the chosen route of synthesis. The person skilled in the art would readily be aware of synthetic possibilities for the synthesis of such compounds.

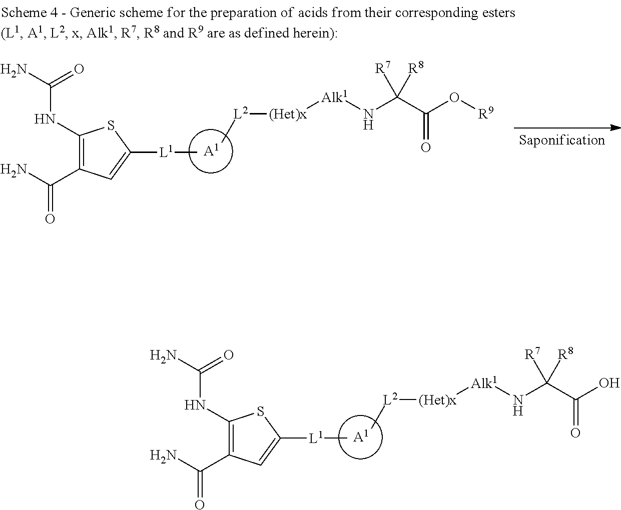

The carboxylic acid derivatives of the esters described herein can be easily prepared from their parent esters by hydrolysis. To the chemist skilled in the art it will be apparent that depending on the ester group to be removed, either basic or acidic conditions may be employed.

### EXAMPLES

The following examples illustrate the preparation and properties of some specific compounds of the invention. The following abbreviations are used:

MeOH=methanol
EtOH=ethanol
EtOAc=ethyl acetate
Boc=tert-butoxycarbonyl
DCE=dichloroethane
DCM=dichloromethane

### DMA=N,N-dimethylacetamide

DME=1,2-dimethoxyethane
DMF=dimethylformamide
DMSO=dimethyl sulfoxide
Bu₄NBr=tetra-butyl ammonium bromide
MeCN=acetonitrile
TFA=trifluoroacetic acid
THF=tetrahydrofuran
Na₂CO₃=sodium carbonate
HCl=hydrochloric acid
NaH=sodium hydride
NaHCO₃=sodium hydrogen carbonate
Pd/C=palladium on carbon
N₂=nitrogen
Na₂SO₄=sodium sulphate
Et₃N=triethylamine
STAB=sodium triacetoxyborohydride
MgSO₄=magnesium sulfate
EDCI=N-(3-Dimethylaminopropyl)-N-ethylcarbodiimide hydrochloride
   Et₂O=diethyl ether
   LiOH=lithium hydroxide

### ELS=Evaporative Light Scattering

TLC=thin layer chromatography
mL=millilitre
g=gram(s)
mg=milligram(s)
mol=moles
mmol=millimole(s)
LCMS=high performance liquid chromatography/mass spectrometry
NMR=nuclear magnetic resonance
RT=room temperature
sat.=saturated aqueous solution
Commercially available reagents and solvents (HPLC grade) were used without further purification. Solvents were removed using a Buchi rotary evaporator or a VirTis Benchtop SLC Freeze-dryer. Microwave irradiation was carried out using a Biotage Initiator™ Eight microwave synthesizer. Specific hydrogenations were carried out using a Thales Technology H-Cube HC-2 continuous hydrogenation equipment. Purification of compounds by flash chromatography column was performed using silica gel, particle size 40-63 μm (230-400 mesh) obtained from Fluorochem. Purification of compounds by preparative HPLC was performed on Gilson systems using reverse phase Axia™ prep Luna C18 columns (10 μm, 100×21.2 mm), gradient 0-100% B (A=water+0.05% TFA, B=acetonitrile) over 10 min, flow=25 mL/min, UV detection at 254 nm.

¹H NMR spectra were recorded on a Bruker 300 MHz AV spectrometer in deuterated solvents. Chemical shifts S are in parts per million. Thin-layer chromatography (TLC) analysis was performed with Kieselgel 60 F₂₅₄ (Merck) plates and visualized using UV light.

Analytical HPLC/MS was performed on an Agilent HP 1100 LC system using reverse phase Luna C18 columns (3 μm, 50×4.6 mm), gradient 5-95% B (A=water+0.1% Formic acid, B=acetonitrile+0.1% Formic acid) over 2.25 min, flow=2.25 mL/min. UV spectra were recorded at 220 and 254 nm using a G1315B DAD detector. Mass spectra were obtained over the range m/z 150 to 800 on a LC/MSD SL G1956B detector. Data were integrated and reported using ChemStation and ChemStation Data Browser softwares.

FIG. 1—The following building blocks were employed in the synthesis of the examples described herein:

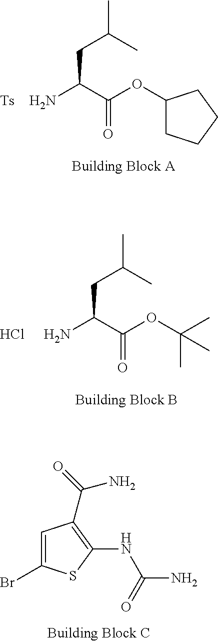

Building Block A—Cyclopentyl L-leucinate was prepared using the methodology outlined in Scheme 1, Route 3:

To a slurry of L-Leucine (5 g, 30.5 mmol) in cyclohexane (150 mL) were added cyclopentanol (27.5 mL, 305 mmol) and p-toluene sulfonic acid (6.33 g, 33.3 mmol). The reaction was fitted with a Dean-Stark receiver and heated to 135° C. for complete dissolution. This temperature was maintained for a period of 12 hours after which time the reaction was complete. The reaction was cooled to RT with precipitation of a white solid. The solid was filtered and washed with EtOAc before drying under reduced pressure. The required product was isolated as the tosylate salt (10.88 g, 85%). m/z=200 [M+H]⁺; ¹H NMR (300 MHz, CD₃OD) δ: 1.01 (6H, t, J=5.8 Hz), 1.54-2.03 (11H, m), 2.39 (3H, s), 3.96 (1H, t, J=6.5 Hz), 5.26-5.36 (1H, m), 7.25 (2H, d, J=7.9 Hz), 7.72 (2H, d, J=8.3 Hz).

Building Block B—tert-Butyl L-leucinate is commercially available (Novabiochem, catalog number: 04-12-5108).

Building Block C—5-Bromo-2-(carbamoylamino)thiophene-3-carboxamide was prepared using the methodology described in WO 03/104218.

### EXAMPLES

### Example 1

### Cyclopentyl N-(4-{[4-carbamoyl-5-(carbamoylamino)-2-thienyl]methyl}benzyl)-L-leucinate

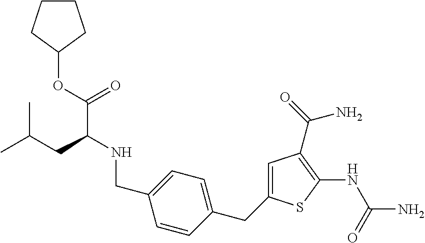

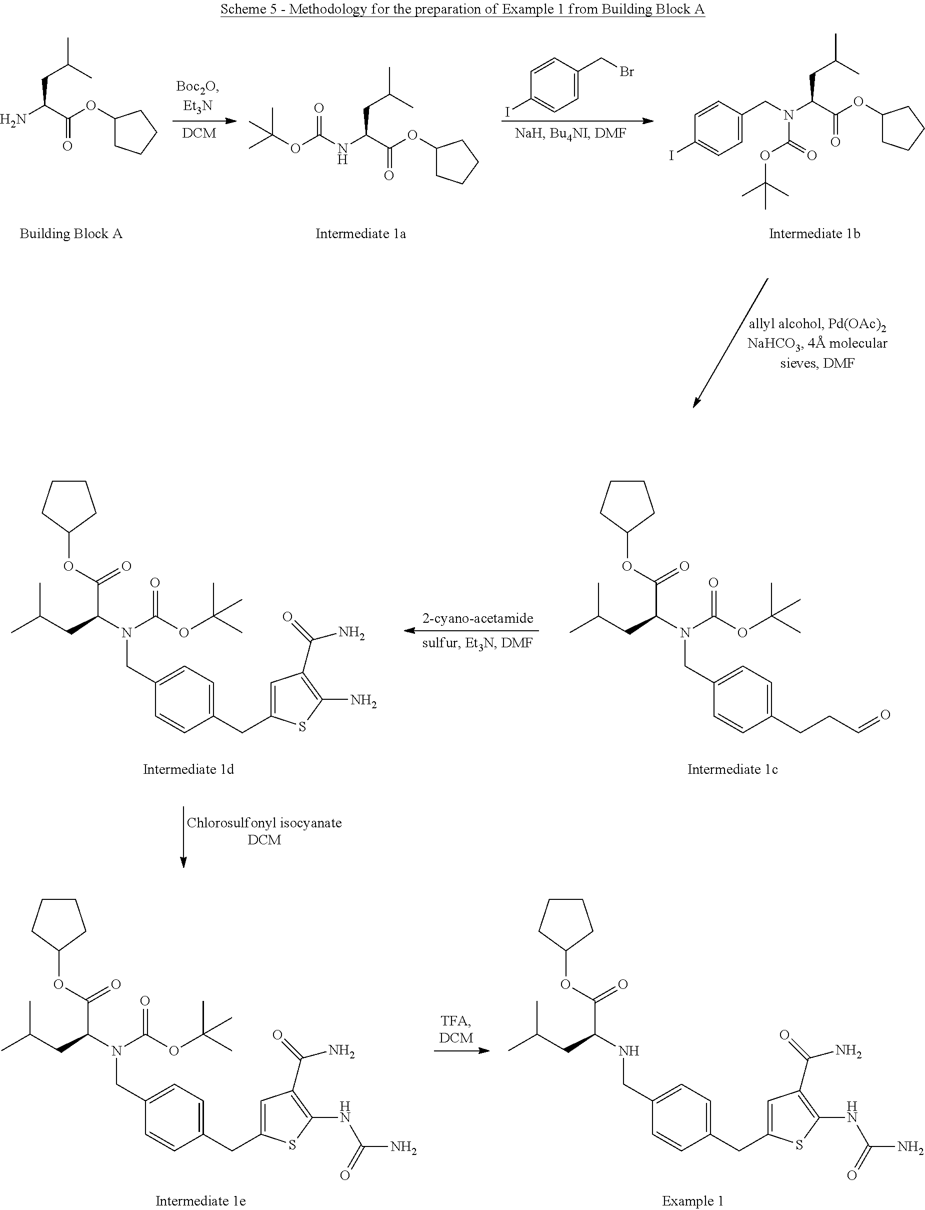

### Intermediate 1a

### Cyclopentyl N-(tert-butoxycarbonyl)-L-leucinate was prepared as follows

To Building Block A (5 g, 13.5 mmol) in DCM (100 mL) was added Et₃N (3.76 mL, 26.9 mmol) and di-tert-butyl dicarbonate (3.24 g, 14.8 mmol). The reaction mixture was stirred at room temperature for 18 hours and then diluted with DCM (100 mL), washing with 1M HCl, sat. NaHCO₃ then brine, dried (MgSO₄) and concentrated under reduced pressure to afford the desired product as a colourless oil, which was taken forward without further purification and characterisation (4 g, 100% yield).

### Intermediate 1b

### Cyclopentyl N-(tert-butoxycarbonyl)-N-(4-iodobenzyl)-L-leucinate was prepared from Intermediate 1a as follows

To Intermediate 1a (1.8 g, 6 mmol)) and Bu₄NI (476 mg, 1.2 mmol) in DMF (50 mL) at 0° C. was added NaH (480 mg, 12 mmol) in DMF (3 mL). The reaction was stirred for 5 minutes before addition of 4-iodo-benzyl bromide (3.56 g, 12 mmol). The reaction mixture was allowed to warm to room temperature and stirred for 4 hours for complete reaction. EtOAc was added (200 mL) and the organic layer washed with water and brine, dried (MgSO₄) and concentrated under reduced pressure. Purification by column chromatography (10% EtOAc/Heptane) afforded the required product as a solid (2.56 g, 82% yield), m/z 516 [M+H]⁺.

### Intermediate 1c

### Cyclopentyl N-(tert-butoxycarbonyl)-N-[4-(3-oxopropyl)benzyl]-L-leucinate was prepared from Intermediate 1b as follows

To Intermediate 1b (2.56 g, 4.9 mmol) in DMF (17 mL) was added Pd(OAc)₂ (34 mg, 0.15 mmol), allyl alcohol (0.51 mL, 7.5 mmol), NaHCO₃ (1.04 g, 12.4 mmol), Bu₄NBr (1.6 g, 4.9 mmol) and 4 Å molecular sieves (965 mg). The reaction mixture was heated at 80° C. for 2 hours. On cooling to room temperature, the mixture was filtered through Celite, washing with DMF and water. The mother liquors were extracted with diethyl ether (2×50 mL) and the organic layer washed with water and brine, dried (MgSO₄) and concentrated under reduced pressure. Purification by column chromatography (30% EtOAc/Heptane) afforded the required product as a solid (1.66 g, 75% yield), m/z 446 [M+H]⁺.

### Intermediate 1d

### Cyclopentyl N-{4-[(5-amino-4-carbamoyl-2-thienyl)methyl]benzyl}-N-(tert-butoxycarbonyl)-L-leucinate was prepared from Intermediate 1c as follows

Triethylamine (0.5 mL, 3.61 mmol) was added dropwise to a mixture of 2-cyano-acetamide (306 mg, 3.61 mmol), sulfur (116 mg, 3.61 mmol) and Intermediate 1c (1.61 g, 3.61 mmol) in DMF (12 mL). The resulting mixture was stirred at room temperature for 18 hours and then poured into water, extracting with EtOAc (2×100 mL). The combined organic layers were washed with brine and dried (MgSO₄) and concentrated under reduced pressure. Purification by column chromatography (50% EtOAc/Heptane) afforded the required product (710 mg, 36% yield), m/z 544 [M+H]⁺.

### Intermediate 1e

### Cyclopentyl N-(tert-butoxycarbonyl)-N-(4-{[4-carbamoyl-5-(carbamoylamino)-2-thienyl]methyl}benzyl)-L-leucinate was prepared from Intermediate 1d as follows

Chlorosulfonyl isocyanate (58 μL, 0.67 mmol) was added dropwise under nitrogen to a solution of Intermediate 1d (300 mg, 0.55 mmol) in anhydrous DCM (6 mL) at 0° C. The reaction mixture was allowed to warm to room temperature and stirred for 3 hours for complete reaction. The reaction was quenched with water (2 mL), stirred for 10 minutes and partitioned between EtOAc and sat NaHCO₃. The organic layer was washed with brine and dried (MgSO₄) and concentrated under reduced pressure. Purification by column chromatography (5% MeOH/DCM) afforded the required product (100 mg, 31% yield), m/z 587 [M+H]⁺.

### Example 1

### Cyclopentyl N-(4-{[4-carbamoyl-5-(carbamoylamino)-2-thienyl]methyl}benzyl)-L-leucinate was prepared from Intermediate 1e as follows

Intermediate 1e (100 mg) was dissolved in DCM (2 mL) and TFA (2 mL) and stirred at room temperature for 18 hours. The reaction mixture was concentrated in vacuo, dissolved in methanol and purified by SCX cation exchange resin. Subsequent purification by Gilson preparative HPLC gave the required product as a light brown solid (60 mg, 72% yield).

LCMS purity=95%; m/z=487 [M+H]⁺; ¹H NMR (300 MHz, CD₃OD) 7.28-7.18 (4H, q, J=8.1 Hz), 6.91 (1H, s), 5.16-5.07 (1H, m), 3.98 (2H, s), 3.73 (1H, d, J=12.9 Hz), 3.63 (1H, d, J=12.9 Hz), 3.24 (1H, t, J=7.3 Hz), 1.94-1.56 (9H, m), 1.51-1.43 (1H, m), 0.91 (3H, d, J=6.6 Hz), 0.85 (3H, d, J=6.6 Hz).

### Example 2

### tert-Butyl N-(4-{[4-carbamoyl-5-(carbamoylamino)-2-thienyl]methyl}benzyl)-L-leucinate

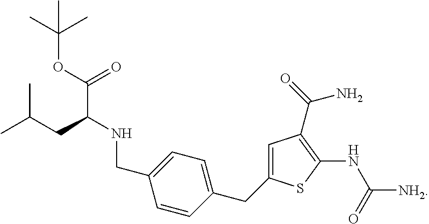

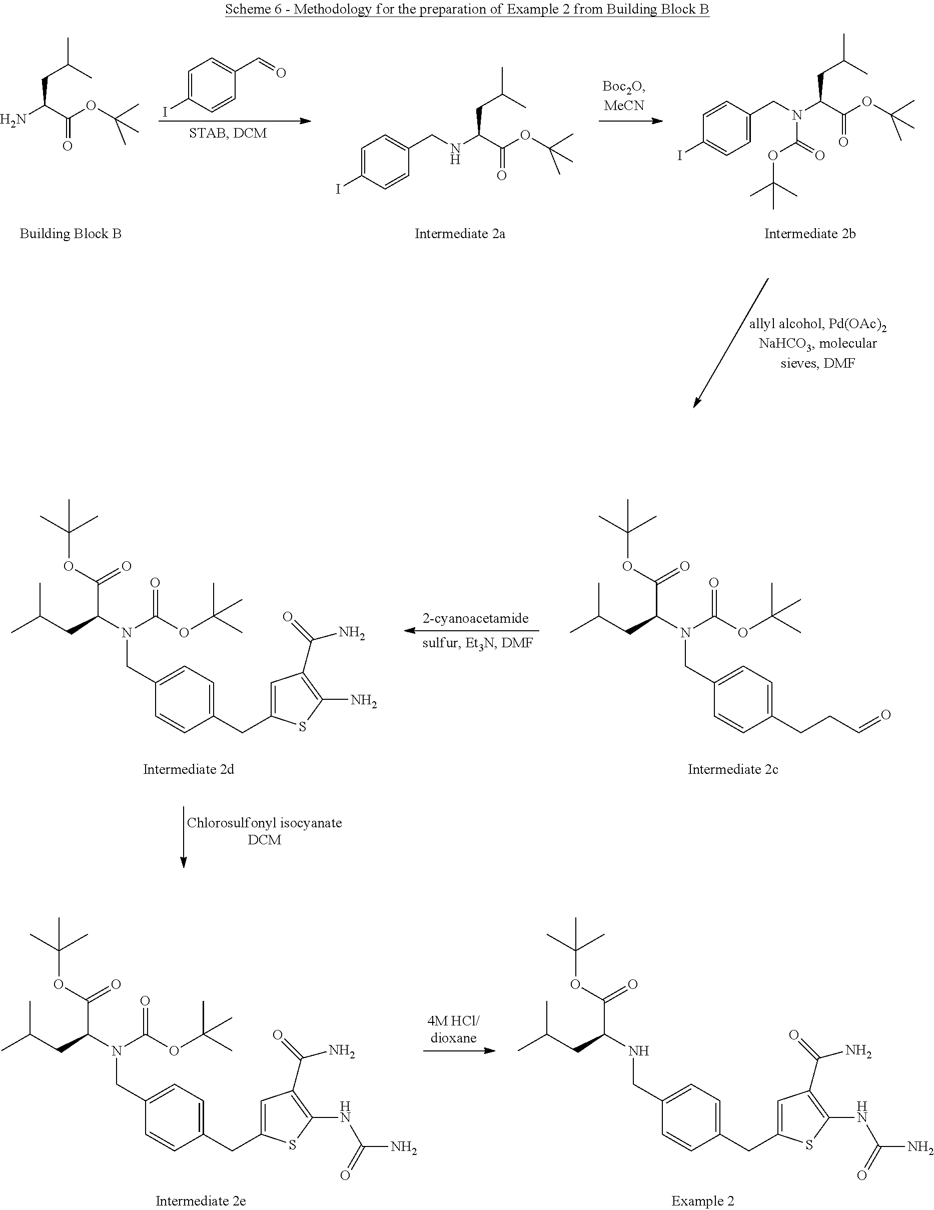

### Intermediate 2a

### tert-Butyl N-(4-iodobenzyl)-L-leucinate was prepared as follows

To Building Block B (2.89 g, 12.9 mmol) in DCM (40 mL) was added 4-iodobenzaldehyde (2 g, 8.63 mmol). The reaction was stirred for 2 hours before cooling to 0° C. and STAB (5.47 g, 25.86 mmol) added portionwise. The reaction was allowed to warm to room temperature and stirred for 18 hours. The reaction mixture was diluted with DCM (100 mL), washing with 1M HCl, sat. NaHCO₃ and brine, dried (MgSO₄) and concentrated under reduced pressure. Purification by column chromatography (20% EtOAc/Heptane) afforded the required product (2.81 g, 80% yield), m/z 404 [M+H]⁺.

### Intermediate 2b

### tert-Butyl N-(tert-butoxycarbonyl)-N-(4-iodobenzyl)-L-leucinate was prepared from Intermediate 2a as follows

To Intermediate 2a (2.8 g 7 mmol) in acetonitrile (30 mL) was added di-tert-butyl dicarbonate (3.08 g, 14.1 mmol). The reaction was allowed to stir at room temperature for 18 hours before concentration under reduced pressure. The crude residue was dissolved in EtOAc and washed with water and brine, dried (MgSO₄) and concentrated under reduced pressure. Purification by column chromatography (3% EtOAc/Heptane) afforded the required product (3.5 g, 100% yield) which was taken forward without further purification and characterization.

Intermediates 2c-2e were prepared using the same methodology as described for Example 1.

### Example 2

### tert-Butyl N-(4-{[4-carbamoyl-5-(carbamoylamino)-2-thienyl]methyl}benzyl)-L-leucinate was prepared from Intermediate 2e as follows

To Intermediate 2e (20 mg) in dioxane (2 mL) at 0° C. under nitrogen was added 4M HCL/dioxane (0.5 mL). The reaction was stirred at 0° C. for 2 hours before quenching with sat NaHCO₃ and concentrated under reduced pressure. The residue was dissolved in methanol and purified by SCX cation exchange resin to give the required product (8 mg). LCMS purity=85%; m/z=475 [M+H]⁺; ¹H NMR (300 MHz, CD₃OD) 7.35 (2H, d, J=8.11Hz), 7.30 (2H, d, J=8.1 Hz), 6.96 (1H, s), 4.03 (2H, s), 3.95-3.80 (2H, m), 2.25-2.16 (1H, m), 1.85-1.65 (2H, m), 1.51 (9H, s), 1.17-1.11 (1H, m), 0.96 (3H, d, J=6.6 Hz), 0.92 (3H, d, J=6.6 Hz).

### Example 3

### Cyclopentyl N-(4-{}2-[4-carbamoyl-5-(carbamoylamino)-2-thienyl]ethyl}benzyl)-L-leucinate

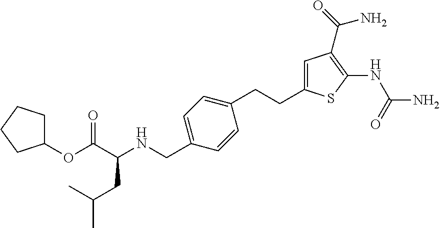

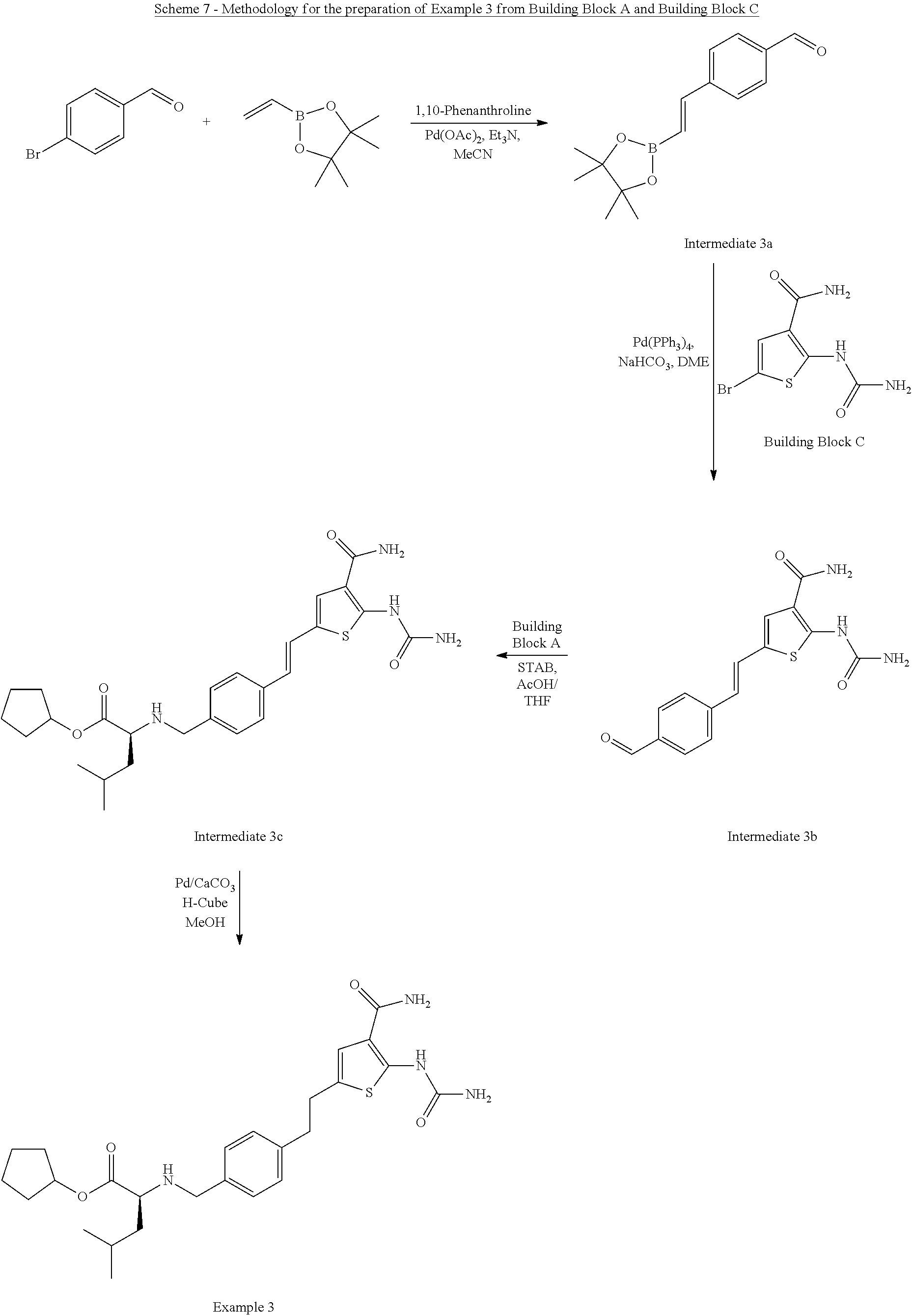

### Intermediate 3a

### 4-[(E)-2-(4,4,5,5-Tetramethyl-1,3,2-dioxaborolan-2-yl)vinyl]benzaldehyde was prepared as follows

A vial was charged with 4-bromobenzaldehyde (1.818 g, 9.83 mmol), palladium acetate (0.110 g, 0.491 mmol), and 1,10-phenanthroline (0.089 g, 0.491 mmol) and subsequently purged with N₂. Anhydrous acetonitrile (15 mL) was added and stirred at room temperature. Triethylamine (2.74 ml, 19.65 mmol) and vinyl boronic acid pinacol ester (2.0 ml, 11.79 mmol) were added and the reaction mixture heated to 60° C. for 18 hours. LCMS analysis after 17 hours showed only 15% product and 75% 4-bromobenzaldehyde starting material. Additional palladium acetate (55 mg) and 1,10-phenanthroline (45 mg) were added as a solution in MeCN and the reaction heated for a further 24 hours at 60° C. The reaction was cooled and 2M HCl added, extracting with diethyl ether. The organic layer was washed with 2M HCl and brine and dried (MgSO₄) and concentrated in vacuo. Purification by column chromatography (50-100% DCM/Hexane gave the required product (0.54 g, 22% yield) as a pale yellow solid, m/z 259 [M+H]⁺.

### Intermediate 3b

### 2-(Carbamoylamino)-5-[(E)-2-(4-formylphenyl)vinyl]thiophene-3-carboxamide was prepared from Intermediate 3a as follows

Intermediate 3a (531 mg, 2.057 mmol) was added to Building Block C (453 mg, 1.714 mmol) and tetrakispalladium (198 mg, 0.171 mmol) and purged with N₂. DME (7 mL) was added along with 1 mL of a saturated aqueous solution of sodium bicarbonate. The mixture was heated at 80° C. for 20 hours for complete reaction. The reaction mixture was cooled and poured into 1M HCl (200 mL) and extracted into EtOAc (300 mL). The combined organic layers were dried (MgSO₄) and concentrated in vacuo to give the product as a yellow solid (342 mg, 60.7% yield), m/z 314 [M−H]⁺.

### Intermediate 3c

### Cyclopentyl N-(4-{(E)-2-[4-carbamoyl-5-(carbamoylamino)-2-thienyl]vinyl}benzyl)-L-leucinate was prepared from Intermediate 3b as follows

Intermediate 3b (249 mg, 0.790 mmol) and Building Block A (157 mg, 0.790 mmol) were dissolved in THF (15 mL) and acetic acid (15 mL) and stirred at room temperature for 30 minutes. STAB (502 mg, 2.369 mmol) was added portionwise and the reaction stirred for 48 hours. 2M HCl was added and the product extracted into EtOAc. The combined organic extracts were washed with water and dried (MgSO₄) and concentrated in vacuo to give 477 mg of an orange oily solid. Purification by column chromatography (2-4% MeOH/DCM) and subsequent SCX cation exchange resin gave the required product (40 mg, 10% yield), m/z 499 [M+H]⁺.

### Example 3

### Cyclopentyl N-(4-{2-[4-carbamoyl-5-(carbamoylamino)-2-thienyl]ethyl}benzyl)-L-leucinate was prepared from Intermediate 3c as follows

Intermediate 3c (40 mg, 0.080 mmol) was dissolved in methanol (6 mL) and pumped through the H-Cube hydrogenator at 3 mL/min using Pd/CaCO₃ (poisoned with Pb) at 1 bar pressure. The methanol was removed under reduced pressure and the crude residue purified by column chromatography (3-5% MeOH/DCM) to give the title compound (15 mg, 38% yield).

LCMS purity=96%; m/z=501 [M+H]⁺; NMR (300 MHz, DMSO-d₆) 10.85 (1H, s), 7.50 (1H, br s), 7.20 (4H, m), 7.15 (1H, br s), 7.01 (1H, s), 6.78 (2H, br s), 5.1 (1H, m), 3.70 (1H, d, J=6.5 Hz), 3.50 (1H, d, J=6.3 Hz), 3.05 (1H, s), 2.23 (1H, m), 1.8 (2H, m), 1.70-1.50 (7H, bm), 1.35 (2H, m), 0.82 (3H, d, J=6.8 Hz), 0.79 (3H, d, J=6.4 Hz).

### Example 4

### Cyclopentyl N-(4-{[4-carbamoyl-5-(carbamoylamino)-2-thienyl]ethynyl}benzyl)-L-leucinate

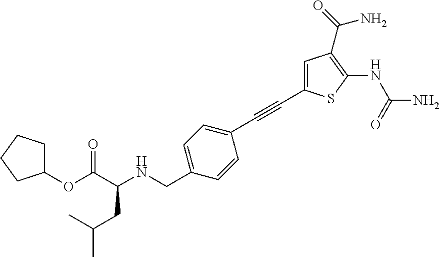

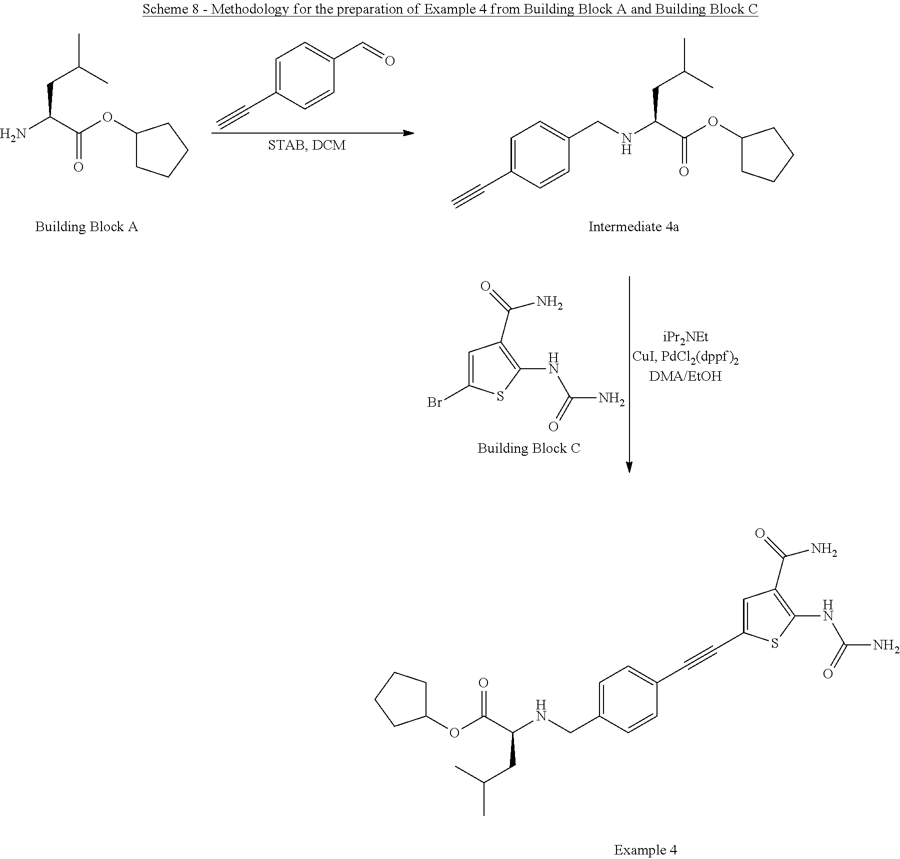

### Intermediate 4a

### Cyclopentyl N-(4-ethynylbenzyl)-L-leucinate

To a solution of Building Block A (918 mg, 4.6 mmol) in DCM (60 mL) was added 4-ethynylbenzaldehyde (500 mg, 3.84 mmol). The reaction was stirred for 3 hours before cooling to 0° C. STAB (2.44 g, 11.5 mmol) was then added portionwise and stirring was continued at room temperature for 18 hours. The reaction was diluted with DCM (50 mL) and washed with 1M HCL (100 mL), sat NaHCO₃ (100 mL), brine (50 mL) and dried (MgSO₄) and concentrated in vacuo. Purification by column chromatography (25% EtOAc/Heptane) gave the required product (0.878 g, 73% yield), m/z 314 [M+H]⁺.

### Example 4

### Cyclopentyl N-(4-{[4-carbamoyl-5-(carbamoylamino)-2-thienyl]ethynyl}benzyl)-L-leucinate was prepared from Intermediate 4a as follows

Intermediate 4a (200 mg, 0.64 mmol) was mixed with Building Block C (91 mg, 0.34 mmol) in DMA (2.5 mL) and EtOH (2.5 mL). Di-isopropylethylamine (0.184 mL, 1.0 mmol) and copper iodide (20 mg, 0.10 mmol) were then added and the solution degassed with nitrogen for 15 minutes. (1,1-Bis(diphenylphosphino)—ferrocene dichloropalladium (8 mg, 8% wt) was then added and the reaction heated to 65° C. for 18 hours. The reaction mixture was cooled and filtered through Celite and purified by Gilson preparative HPLC. The required product was isolated as a white solid (17 mg, 10% yield).

LCMS purity=90%; m/z=497 [M+H]⁺; ¹H NMR (300 MHz, CD₃OD) 7.61 (2H, d, J=6.5 Hz), 7.55 (2H, d, J=6.8 Hz), 7.45 (1H, s), 5.31 (1H, m), 4.25 (2H, m), 4.05 (1H, m), 2.05-1.65 (11H, bm), 1.00 (6H, m).

### Example 5

### N-(4-{[4-Carbamoyl-5-(carbamoylamino)-2-thienyl]methyl}benzyl)-L-leucine

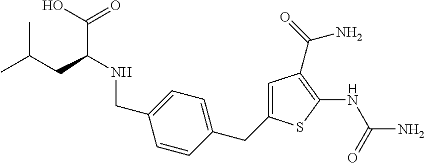

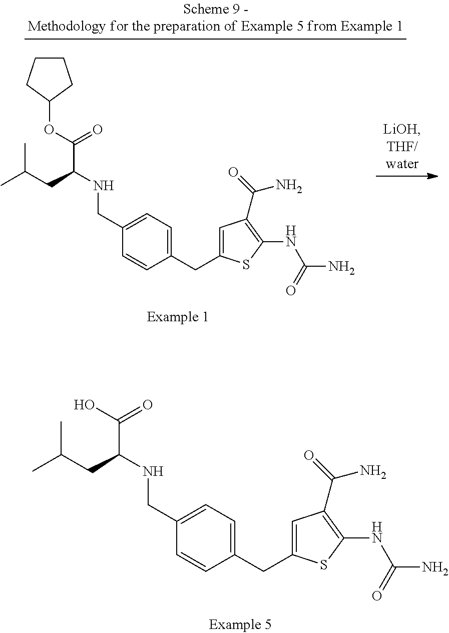

### Example 5

### N-(4-{[4-Carbamoyl-5-(carbamoylamino)-2-thienyl]methyl}benzyl)-L-leucine was prepared from Example 1 as follows

To a solution of Example 1 (50 mg, 0.1 mmol) in THF (2 mL) and water (2 mL) was added lithium hydroxide (25 mg, 1 mmol). The reaction mixture was heated at 40° C. for 18 hours for complete reaction. The reaction was cooled to room temperature and the THF removed under reduced pressure before diluting with water and adjusting the pH to 4 with acetic acid. The precipitate was filtered and washed with water and diethyl ether before purification by Gilson preparative HPLC. The required product was isolated as a white solid (11 mg, 26% yield).

LCMS purity=94%; m/z=419 [M+H]⁺; ¹H NMR (300 MHz, DMSO-d₆) 10.88 (1H, s), 7.58 (1H, br s), 7.33 (2H, d, J=7.8 Hz), 7.23 (2H, d, J=7.8 Hz), 7.16 (1H, br s), 7.06 (1H, s), 6.83 (2H, br s), 3.94 (2H, s), 3.90 (1H, d, J=13.2 Hz), 3.75 (1H, d, J=13.2 Hz), 3.13 (1H, t, J=7.1 Hz), 1.90-1.70 (1H, m), 1.52-1.40 (2H, m), 0.85 (3H, d, J=6.6 Hz), 0.79 (3H, d, J=6.6 Hz).

### Example 6

### N-(4-{2-[4-Carbamoyl-5-(carbamoylamino)-2-thienyl]ethyl}benzyl)-L-leucine

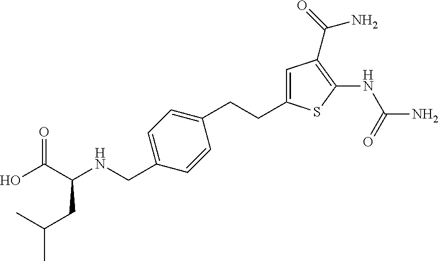

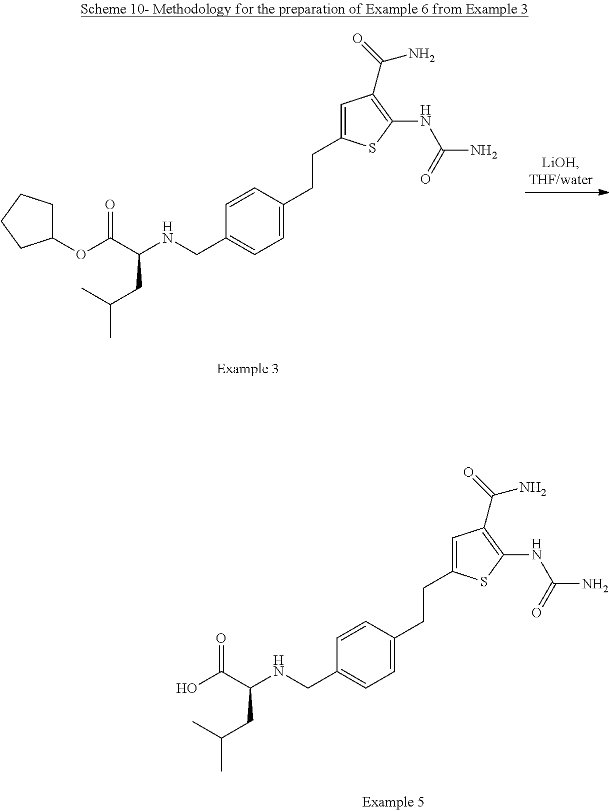

Example 6 was prepared using the same methodology as described for Example 5.

LCMS purity=97%; m/z=431 [M−H]⁺; ¹H NMR (300 MHz, DMSO-d₆) 10.85 (1H, s), 7.49 (1H, br s), 7.22 (2H, d, J=7.8 Hz), 7.15 (2H, d, J=7.8 Hz), 7.10 (1H, br s), 6.99 (1H, s), 6.75 (2H, br s), 3.75 (1H, d, J=13.2 Hz), 3.50 (1H, d, J=13.2 Hz), 3.13 (1H, m), 1.75 (1H, m), 1.40-1.20 (3H, bm), 0.83 (3H, d, J=6.6 Hz), 0.73 (3H, d, J=6.6 Hz).

### Biological Assays

### IKKβ Enzyme Assay

The ability of compounds to inhibit IKKβ kinase activity was measured in an assay performed by Invitrogen (Paisley, UK). The Z′-LYTE™ biochemical assay employs a fluorescence-based, coupled-enzyme format and is based on the differential sensitivity of phosphorylated and non-phosphorylated peptides to proteolytic cleavage. The peptide substrate is labelled with two fluorophores—one at each end—that make up a FRET pair. In the primary reaction, the kinase transfers the gamma-phosphate of ATP to a single serine or threonine residue in a synthetic FRET-peptide. In the secondary reaction, a site-specific protease recognizes and cleaves non-phosphorylated FRET-peptides. Phosphorylation of FRET-peptides suppresses cleavage by the Development Reagent. Cleavage disrupts FRET between the donor (i.e., coumarin) and acceptor (i.e. fluorescein) fluorophores on the FRET-peptide, whereas uncleaved, phosphorylated FRET-peptides maintain FRET. A radiometric method, which calculates the ratio (the Emission Ratio) of donor emission to acceptor emission after excitation of the donor fluorophore at 400 nm, is used to quantitate reaction progress.

The final 10 μL Kinase Reaction consists of 0.9-8.0 ng IκBKB (IKKβ), 2 μM Ser/Thr 05 Peptide and ATP in 50 mM HEPES pH 7.5, 0.01% BRIJ-35, 10 mM MgCl₂, 1 mM EGTA. The assay is performed at an ATP concentration at, or close to the Km. After the 60 minute Kinase Reaction incubation at room temperature, 5 μL of a 1:128 dilution of Development Reagent is added. The assay plate is incubated for a further 60 minutes at room temperature and read on a fluorescence plate reader.

Duplicate data points are generated from a 1/3 log dilution series of a stock solution of test compound in DMSO. Nine dilutions steps are made from a top concentration of 10 μM, and a ‘no compound’ blank is included. Data is collected and analysed using XLfit software from IDBS. The dose response curve is curve fitted to model number 205 (sigmoidal dose-response model). From the curve generated, the concentration giving 50% inhibition is determined and reported.

### LPS-Stimulation of THP-1 Cells

THP-1 cells were plated in 100 μl at a density of 4×10⁴ cells/well in V-bottomed 96 well tissue culture treated plates and incubated at 37° C. in 5% CO₂ for 16 hours. 2 hours after the addition of the inhibitor in 100 μl of tissue culture media, the cells were stimulated with LPS (E coli strain 005:B5, Sigma) at a final concentration of 1 μg/ml and incubated at 37° C. in 5% CO₂ for 6 hrs. TNF-α levels were measured from cell-free supernatants by sandwich ELISA (R&D Systems #QTA00B).

### LPS-Stimulation of Human Whole Blood

Whole blood was taken by venous puncture using heparinised vacutainers (Becton Dickinson) and diluted in an equal volume of RPMI1640 tissue culture media (Sigma). 100 μl was plated in V-bottomed 96 well tissue culture treated plates. 2 hours after the addition of the inhibitor in 100 μlof RPMI1640 media, the blood was stimulated with LPS (E coli strain 005:B5, Sigma) at a final concentration of 100 ng/ml and incubated at 37° C. in 5% CO₂ for 6 hours. TNF-α levels were measured from cell-free supernatants by sandwich ELISA (R&D Systems #QTA00B).

### Broken Cell Assay

In order to determine whether a compound containing a particular group R² is hydrolysable by one or more intracellular carboxylesterase enzymes to a —COOH group, the compound may be tested in the following assay:

### Preparation of Cell Extract

U937 or HUT78 tumour cells (−109) are washed in 4 volumes of Dulbeccos PBS (˜1 litre) and pelleted at 525 g for 10 minutes at 4° C. This is repeated twice and the final cell pellet is resuspended in 35 ml of cold homogenising buffer (Trizma 10 mM, NaCl-130 mM, CaCl₂ 0.5 mM pH 7.0 at 25° C.). Homogenates are prepared by nitrogen cavitation (700 psi for 50 minutes at 4° C.). The homogenate is kept on ice and supplemented with a cocktail of inhibitors at final concentrations of Leupeptin Aprotinin 0.1 μM, E64 8 μM, Pepstatin 1.5 μM, Bestatin 162 μM, Chymostatin 33 μM. After clarification of the cell homogenate by centrifugation at 1500 rpm for 10 minutes, the resulting supernatant is used as a source of esterase activity and is stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 ml) is incubated at 37° C. in a Tris-HCl 25 mM, 125 mM NaCl buffer, pH 7.5 at 25° C. At zero time the ester (substrate) is then added at a final concentration of 2.5 mM and the samples were incubated at 37° C. for the appropriate time (usually 0 or 80 minutes). Reactions are stopped by the addition of 2× volumes of acetonitrile. For zero time samples the acetonitrile is added prior to the ester compound. After centrifugation at 12000 g for 5 minutes, samples are analysed for the ester and its corresponding carboxylic acid at room temperature by LCMS (Sciex API 3000, HP1100 binary pump, CTC PAL). Chromatography was based on a MeCN (75×2.1 mm) column and a mobile phase of 5-95% acetonitrile in water/0.1% formic acid. Rates of hydrolysis are expressed in pg/mL/min.

### Intact Cell Accumulation Assay

Cells (4×10⁴/mL) were incubated at 37° C. in culture medium containing 6 μmol/L compound in a 5% (v/v) CO₂-humidified atmosphere. Incubations were terminated after 6 h by centrifugation of 25 mL aliquots of the cell suspension at 1500 rpm for 5 min at 4° C. 0.2 mL samples of the culture media supernatants were added to four volumes of acetonitrile. After decanting the supernatant, the residual cell pellet (10⁶ cells) was extracted into 1 mL of acetonitrile. Samples were then analyzed for the ester and acid metabolite at room temperature by LC/MS/MS (Sciex API3000). Chromatography was based on a MeCN (75×21 mm) column with a 5% to 95% (v/v) acetonitrile/water, 0.1% (v/v) formic acid mobile phase. For the zero time samples, the cell suspension was chilled and centrifuged as soon as the ester had been added and then extracted into acetonitrile as described. Levels in cells are expressed as ng/mL.

### Results:

IC₅₀ values obtained in the biological assays for the exemplary compounds of the invention are presented in Table 1. IC₅₀ values are allocated to one of three ranges as follows:

### Range A: IC₅₀<500 nM

### Range B: 500 nM <C₅₀<5000 nM

### Range C: IC₅₀>5000 nM

TABLE 1

| | | IC50 (nM) | | |
|---|---|---|---|---|
| | | | | Human |
| | | IKKβ | | Whole |
| Example | Name | Enzyme | THP-1 | Blood |
| 1 | Cyclopentyl N-(4-{[4-carbamoyl-5- | C | B | A |
| | (carbamoylamino)-2-thienyl]methyl}benzyl)- | | | |
| | L-leucinate | | | |
| 2 | tert-Butyl N-(4-{[4-carbamoyl-5- | B | C | C |
| | (carbamoylamino)-2-thienyl]methyl}benzyl)- | | | |
| | L-leucinate | | | |
| 3 | Cyclopentyl N-(4-{2-[4-carbamoyl-5- | C | C | B |
| | (carbamoylamino)-2-thienyl]ethyl}benzyl)-L- | | | |
| | leucinate | | | |
| 4 | Cyclopentyl N-(4-{[4-carbamoyl-5- | C | B | B |
| | (carbamoylamino)-2-thienyl]ethynyl}benzyl)- | | | |
| | L-leucinate | | | |
| 5 | N-(4-{[4-Carbamoyl-5-(carbamoylamino)-2- | A | NR | NR |
| | thienyl]methyl}benzyl)-L-leucine | | | |
| 6 | N-(4-{2-[4-Carbamoyl-5-(carbamoylamino)- | B | NR | NR |
| | 2-thienyl]ethyl}benzyl)-L-leucine | | | |

NT = Not Tested.
NR = Not Relevant. Examples 5-6 are the resultant carboxylic acid analogues of the amino acid esters that are cleaved inside cells. When these carboxylic acids are contacted with the cells, they do not penetrate into the cells and hence do not inhibit TNF-α production in these assays.

Concentrations of the compounds of Examples 1 and 5 associated with U937 (monocyte cell line) cells and HUT78 (non-monocyte cell line) cells after 0 and 6 hour incubation times are presented in Table 2.

TABLE 2

| | Concentration in U937 cell | | Concentration in HUT78 cell | |
|---|---|---|---|---|
| | line (ng/10⁶ cells) | | line (ng/10⁶ cells) | |
| Example | 0 hrs | 6 hrs | 0 hrs | 6 hrs |
| 1 | 420 | 217 | 466 | 465 |
| 5 | 68 | 286 | 0 | 0 |

Table 2 shows that the compound of Example 1 (ester derivative) is able to accumulate in both U937 and HUT78 cell lines in concentrations of 420 ng/10⁶ cells and 466 ng/10⁶ cells respectively at the 0 hour time point. After 6 hours incubation, it becomes clear that the compound of Example 5 (acid derivative) is selectively accumulating in the U937 monocytic cell line (268 ng/10⁶ cells) with no accumulation in the corresponding HUT78 non-monocytic cell line. The compound of Example 1 is being hydrolysed by human carboxylesterase hCE-1 (only present in macrophage-related cell lines such as U937) and hence the compound of Example 4 will selectively accumulate in macrophage-related cells. The concentration of the compound of Example 1 in HUT78 at 6 hours remains unchanged to the 0 hour time point (466 vs 465 ng/10⁶ cells). The small amount of Example 5 associated with the U937 cells at 0 hour time point reflects acid formed within the cell during the separation of cells and medium.

A comparison between in vitro biological data of the compound of Example 1 with its parent molecule (Compound I) is presented in Table 3.

TABLE 3

| | | | Inhibition of | Inhibition of | Ratio of | Cell |
|---|---|---|---|---|---|---|
| | | | TNF-α | TNF-α | whole | accumulation |
| | Inhibition | | production | production in | blood IC₅₀ | in U937 cells |
| | of IKKβ | | in THP-1 | human whole | to enzyme | is at 6 hours |
| Compound | (IC₅₀ nM) | | cells (IC₅₀, nM) | blood (IC₅₀, nM) | IC₅₀ | (ng/mL) |
| 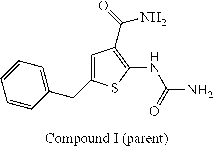 | 486 | | 2711 | 28% @10 μM | >20 | NA |
| 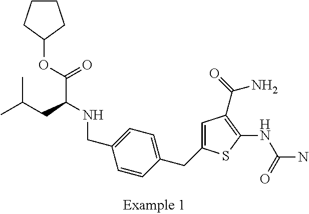 | Ester 10,000 | Acid 369 | 1936 | 497 | 0.05 | 268 |

Table 3 shows that the acid of the compound of Example 1 has a similar IC₅₀ in the enzyme assay to its parent compound (Compound I), indicating that binding to the enzyme has not been disrupted by attachment of the esterase motif. Methylene-bridged compounds (i.e., L¹ is a methylene group) such as the compound of Example 1 are hydrolysed by hCE-1 and as a consequence the acid accumulates in cells. This accumulation of acid results in the compound of Example 1 being significantly more potent than the parent compound, particularly in the human whole blood assay. These data highlight the potency benefit that can be achieved by the attachment of the esterase motif and the resulting cellular accumulation of the corresponding acid.

1. A compound which is: (a) a thiophene carboxamide derivative of formula (IA) or (IB), or a tautomer thereof; or (b) a pharmaceutically acceptable salt, N-oxide, hydrate or solvate thereof: 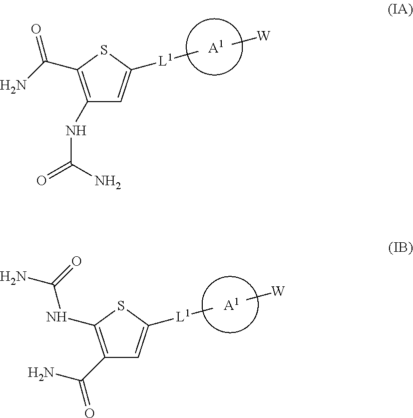
wherein:
L¹ represents a C₁₋₄ alkylene, C₂₋₄ alkenylene or C₂₋₄ alkynylene group, or L¹ represents a group —(CH₂)ₘ(C═O)NR¹(CH₂)ₙ—, —(CH₂)ₘNR¹(C═O)(CH₂)ₙ—, —(CH₂)ₘO(CH₂)ₙ—, —(CH₂)ₘS(CH₂)ₙ—, —(CH₂)ₘ(C═O)(CH₂)ₙ— or —(CH₂)ₘNR¹(CH₂)ₙ—, in which R¹ is C₁₋₄ alkyl and m and n are the same or different and are 0, 1, 2, 3 or 4;
ring A¹ is a C₆₋₁₀ aryl, 5- to 10-membered heteroaryl, C₃₋₇ carbocyclyl or 5- to 10-membered heterocyclyl group which is optionally fused to a further C₆₋₁₀ aryl, 5- to 10-membered heteroaryl, C₃₋₇ carbocyclyl or 5- to 10-membered heterocyclyl group;
W represents a group of formula:
-L²-(Het)ₓ-Alk¹-R
wherein:
L² represents a group -Alk²-, -Alk²-A²- or -Alk²-Alk³-;
Alk² represents a bond or a C₁₋₄ alkylene, C₂₋₄ alkenylene or C₂₋₄ alkynylene group;
Alk³ represents a C₁₋₄ alkylene, C₂₋₄ alkenylene or C₂₋₄ alkynylene group;
A² represents a phenyl or 5- to 6-membered heteroaryl group which is unfused or fused to a further phenyl or 5- to 6-membered heteroaryl group;
Het represents —O—, —S— or —NR′— where R¹ represents hydrogen or unsubstituted C₁₋₂ alkyl;
x is 0 or 1;
Alk¹ represents a bond or a C₁₋₆ alkylene, C₂₋₆ alkenylene or C₂₋₆ alkynylene group, or a group -A³-Alk⁴- in which A³ represents a phenyl or 5- to 6-membered heteroaryl group which is unfused or fused to a further phenyl or 5- to 6-membered heteroaryl group, and Alk⁴ represents a bond or a C₁₋₆ alkylene, C₂₋₆ alkenylene or C₂₋₆ alkynylene group;
R represents a group of formula (X1), (X2), (Y1) or (Y2): 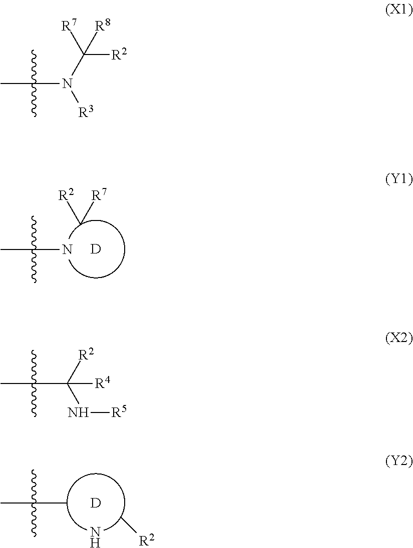
in which
R² is a group —COOH or an ester group which is hydrolysable by one or more intracellular carboxylesterase enzymes to a —COOH group;
R³ represents a hydrogen atom or a C₁₋₄ alkyl group;
R⁴, R⁷ and R⁸ are the same or different and each represents the α-substituent of a natural or non-natural α-amino acid, or R⁷ and R⁸, taken together with the carbon to which they are attached, form a 3- to 6-membered saturated spiro cycloalkyl or heterocyclyl ring;
R⁵ represents a hydrogen atom or a C₁₋₆ alkyl, C₃₋₇ carbocyclyl, C₆₋₁₀ aryl or 5- to 6-membered heteroaryl group, or a group of formula —(C═O)R⁶, —(C═O)OR⁶, or —(C═O)NR⁶ wherein R⁶ is a hydrogen atom or a C₁₋₆ alkyl group; and
ring D is a 5- to 6-membered heterocyclyl group wherein: R² is linked to a ring carbon adjacent to the ring nitrogen shown; R⁷, if present, is linked to the same ring carbon as R²; and ring D is optionally fused to a second ring comprising a phenyl, 5- to 6-membered heteroaryl, C₃₋₇ carbocyclyl or 5- to 6-membered heterocyclyl group;
with the proviso that if R represents a group of formula (Y2) and the ring D is fused to a second ring comprising a phenyl, 5- to 6-membered heteroaryl, C₃₋₇ carbocyclyl or 5- to 6-membered heterocyclyl group then the bond shown intersected by a wavy line may be from a ring atom in ring D or said second ring;
and wherein, unless otherwise stated:
any alkyl, alkenyl and alkynyl groups and moieties in R¹, R², R³, R⁴, R⁵, R⁶, R⁷, R⁸, L¹, Alk¹, Alk², Alk³ and Alk⁴ are the same or different and are each unsubstituted or substituted with 1, 2 or 3 unsubstituted substituents which are the same or different and are selected from halogen atoms and C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ haloalkoxy, C₂₋₄ haloalkenyloxy, hydroxyl, —SR′, cyano, nitro, C₁₋₄ hydroxyalkyl and —NR′R″ groups where R′ and R″ are the same or different and represent hydrogen or unsubstituted C₁₋₂ alkyl; and
any aryl, heteroaryl, carbocyclyl and heterocyclyl groups and moieties in A¹, A², A³, D and R⁵ are the same or different and are each unsubstituted or substituted by 1, 2, 3 or 4 unsubstituted substituents selected from halogen atoms, and cyano, nitro, C₁₋₄ alkyl, C₁₋₄ alkoxy, C₂₋₄ alkenyl, C₂₋₄ alkenyloxy, C₁₋₄ haloalkyl, C₂₋₄ haloalkenyl, C₁₋₄ haloalkoxy, C₂₋₄ haloalkenyloxy, hydroxyl, C₁₋₄ hydroxyalkyl, —SR′ and —NR′R″ groups wherein each R′ and R″ is the same or different and represents hydrogen or unsubstituted C₁₋₄ alkyl, or from substituents of formula —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —OCONH₂, —OCONHR^{A}, —OCONR^{A}R^{B}, —NHCOR^{A}, —NR^{B}COR^{A}, —NHCOOR^{A}, —NR^{B}COOR^{A}, —NR^{B}COOH, —NHCOOH, —NHSO₂R^{A}, —NR^{B}SO₂R^{A}, —NHSO₂OR^{A}, —NR^{B}SO₂OH, —NHSO₂H, —NR^{B}SO₂OR^{A}, —NHCONH₂, —NR^{A}CONH₂, —NHCONHR^{B}, —NR^{A}CONHR^{B}, —NHCONR^{A}R^{B} or —NR^{A}CONR^{A}R^{B} wherein R^{A} and R^{B} are the same or different and represent unsubstituted C₁₋₆ alkyl, C₃₋₆ cycloalkyl, non-fused phenyl or a non-fused 5- to 6-membered heteroaryl, or R^{A} and R^{B} when attached to the same nitrogen atom form a non-fused 5- or 6-membered heterocyclyl group.

2. A compound as claimed in claim 1 wherein:
R⁴, R⁷ and R⁸ are the same or different and each represents different and each represents:
(i) a hydrogen atom;
(ii) a C₁₋₆ alkyl group;
(iii) a group -L³-B, in which L³ represents a bond or a C₁₋₆ alkylene group and B represents a C₆₋₁₀ aryl or 5- to 10-membered heteroaryl group; or
(iv) a group selected from indol-3-ylmethyl, —CH₂COOH, —CH₂CH₂COOH, —CH₂CONH₂, —CH₂CH₂CONH₂, —CH₂CH₂CH₂NHC(NH)NH₂, cyclohexyl, cyclohexylmethyl and 1-benzylthio-1-methylethyl;
said C₁₋₆ alkyl group in (ii) is unsubstituted or substituted with 1 or 2 substituents which are the same or different and represent halogen, C₁₋₂ alkoxy, C₁₋₂ haloalkyl, hydroxyl, —COOR′, —COONR′R″, —SR′ and —NR′R″ wherein R′ and R″ are the same or different and represent hydrogen or C₁₋₂ alkyl;
said C₁₋₆ alkylene group in (iii) is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl; and
said C₆₋₁₀ aryl or 5- to 10-membered heteroaryl group in (iii) is unsubstituted or substituted by 1, 2 or 3 substituents which are the same or different and are selected from halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups where R′ and R″ are the same or different and represent hydrogen or unsubstituted C₁₋₂ alkyl.

3. A compound as claimed in claim 1 or 2 wherein:
L¹ represents —O—, —S—, —NR′— or C₁₋₄ alkylene, wherein R¹ represents unsubstituted C₁₋₄ alkyl and the C₁₋₄ alkylene moiety is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl.

4. A compound as claimed in any one of the preceding claims wherein:
L¹ represents —O— or C₁₋₄ alkylene wherein the C₁₋₄ alkylene moiety is unsubstituted or substituted with 1 or 2 unsubstituted substituents which are the same or different and are selected from halogen atoms and C₁₋₂ alkoxy, hydroxyl, C₁₋₂ haloalkyl and —NR′R″ groups wherein R′ and R″ are the same or different and represent hydrogen or unsubstituted C₁₋₂ alkyl.

5. A compound as claimed in any one of the preceding claims wherein:
A¹ represents a phenyl or 5- to 6-membered heteroaryl group which is unfused or fused to a further phenyl or 5- to 6-membered heterocyclyl group, said A¹ group being unsubstituted or substituted by 1, 2 or 3 substituents which are the same or different and are selected from halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups where R′ and R″ are the same or different and represent hydrogen or unsubstituted C₁₋₂ alkyl.

6. A compound as claimed in any one of the preceding claims wherein:
Alk² represents a bond or a C₁₋₃ alkylene, C₂₋₃ alkenylene or C₂₋₃ alkynylene group which is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups wherein R′ and R″ are the same or different and represent hydrogen or unsubstituted C₁₋₂ alkyl;
Alk³ represents a C₁₋₄ alkylene, C₂₋₄ alkenylene or C₂₋₄ alkynylene group which is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups wherein R′ and R″ are the same or different and represent hydrogen or unsubstituted C₁₋₂ alkyl; and
A² represents an unfused phenyl or unfused 5- to 6-membered heteroaryl group which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl.

7. A compound as claimed in any one of the preceding claims wherein:
Het represents —O—, —NR′ or —S—, wherein R¹ represents hydrogen or unsubstituted methyl;
Alk¹ represents a bond or a C₁₋₆ alkylene group which is unsubstituted or substituted with 1, 2 or 3 unsubstituted substituents selected from halogen atoms and C₁₋₄ alkoxy, hydroxyl, C₁₋₄ haloalkyl, C₂₋₄ haloalkenyl, C₁₋₄ haloalkoxy and —NR′R″ groups wherein R′ and R″ are the same or different and represent hydrogen or unsubstituted C₁₋₂ alkyl, or Alk¹ represents a group -A³-Alk⁴- wherein A³ represents an unfused phenyl or unfused 5- to 6-membered heteroaryl group which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″′ groups wherein R′ and R″ are the same or different and represent hydrogen or unsubstituted C₁₋₂ alkyl, and Alk⁴ represents a bond or an C₁₋₃ alkylene, C₂₋₃ alkenylene or C₂₋₃ alkynylene group which is unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups wherein R′ and R″ are the same or different and represent hydrogen or unsubstituted C₁₋₂ alkyl;
R³ represents a hydrogen atom or an unsubstituted C₁₋₂ alkyl group;
R⁴, R⁷ and R⁸, which are the same or different, represent: a hydrogen atom; a C₁₋₆ alkyl group which is unsubstituted or substituted with 1 or 2 substituents which are the same or different and represent halogen, C₁₋₂ alkoxy, C₁₋₂ haloalkyl, hydroxyl, —COOR′, —COONR′R″, —SR′ and —NR′R″ wherein R′ and R″ are the same or different and represent hydrogen or C₁₋₂ alkyl; or a group of formula -L³-B wherein L³ is a bond or an unsubstituted C₁₋₄ alkylene group and B represents a phenyl or a 5- to 10-membered heteroaryl group which is unsubstituted or substituted with one, two or three unsubstituted substituents which are the same or different and represent halogen atoms, C₁₋₄ alkyl, C₁₋₂ alkoxy, C₁₋₂ alkylthio and hydroxy;
R⁵ represents a hydrogen atom or an unsubstituted C₁₋₄ alkyl group, or a group of formula —(C═O)R⁶, —(C═O)OR⁶, or —(C═O)NR⁶ wherein R⁶ is a hydrogen atom or an unsubstituted C₁₋₄ alkyl group; and
Ring D represents an unfused 5- to 6-membered heterocyclyl group.

8. A compound as claimed in any one of the preceding claims wherein:
L¹ represents C₁₋₄ alkylene which is unsubstituted or substituted with 1 or 2 unsubstituted substituents which are the same or different and are selected from halogen atoms and C₁₋₂ alkoxy and C₁₋₂ haloalkyl groups;
A¹ represents an unfused phenyl or a 5- to 6-membered heteroaryl group which is unsubstituted or substituted by 1, 2 or 3 substituents which are the same or different and are selected from halogen atoms and unsubstituted C₁₋₄ alkyl, C₁₋₄ alkoxy, hydroxyl, C₁₋₄ haloalkyl, C₁₋₄ haloalkoxy, C₁₋₄ hydroxyalkyl, cyano, nitro, —SR′ and —NR′R″ groups wherein R′ and R″ are the same or different and represent hydrogen or unsubstituted C₁₋₂ alkyl;
Alk² represents an unsubstituted C₁₋₃ alkylene, C₂₋₃ alkenylene or C₂₋₃ alkynylene group;
Alk³ represents an unsubstituted C₁₋₄ alkylene group;
A² represents an unfused phenyl which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl;
Het represents —O—, —NH— or —S—;
Alk¹ represents a bond or a C₁₋₄ alkylene group which is unsubstituted or substituted with 1 or 2 unsubstituted substituents selected from halogen atoms and C₁₋₂ alkoxy, hydroxyl, C₁₋₂ haloalkyl and —NR′R″ groups where R′ and R″ are the same or different and represent hydrogen or unsubstituted C₁₋₂ alkyl, or Alk¹ represents a group -A³-Alk⁴- where A³ represents an unfused phenyl or unfused 5- to 6-membered heteroaryl group which is unsubstituted or substituted with 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl, and Alk⁴ represents an unsubstituted C₁₋₃ alkylene group;
R represents a group of formula (X1) or (Y1): 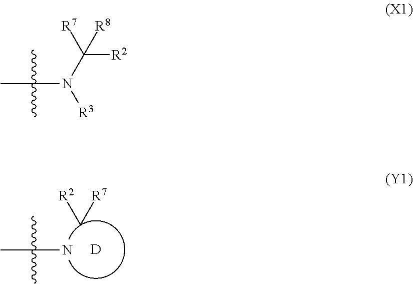
in which
R² represents —COOH or —COOR⁹ wherein R⁹ represents a C₁₋₄ alkyl, C₃₋₇ carbocyclyl or C₂₋₄ alkenyl group, or R⁹ represents a phenyl, benzyl, 2-pyridylmethyl, 3-pyridylmethyl, 4-pyridylmethyl, N-methylpiperidin-4-yl, tetrahydrofuran-3-yl, methoxyethyl, indanyl, norbonyl, dimethylaminoethyl or morpholinoethyl group, said R⁹ being unsubstituted or substituted with 1, 2 or 3 unsubstituted substituents which are the same or different and are selected from halogen atoms and C₁₋₂ alkoxy, hydroxyl, C₁₋₂ haloalkyl and —NR′R″ groups where R′ and R″ are the same or different and represent hydrogen or unsubstituted
C₁₋₂ alkyl;
R³ represents a hydrogen atom or an unsubstituted methyl group;
R⁷ and R⁸, which are the same or different, represent a hydrogen atom or an unsubstituted C₁₋₆ alkyl group; and
Ring D represents an unfused unsubstituted 5- to 6-membered heterocyclyl group.

9. A compound as claimed in claim 8 wherein:
L¹ represents unsubstituted C₁₋₄ alkylene.

10. A compound as claimed in claim 8 or 9 wherein:
A¹ represents a 1,4-phenylene or 1,3-phenylene group, which is unsubstituted or substituted by 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl.

11. A compound as claimed in any one of claims 8 to 10 wherein:
x is 0;
L² represents the group -Alk²-; and
Alk¹ represents a bond.

12. A compound as claimed in any one of claims 8 to 11 wherein R represents a group of formula (X1).

13. A compound as claimed in any one of claims 8 to 12 wherein:
R² represents —COOR⁹ wherein R⁹ represents an unsubstituted C₁₋₄ alkyl, C₃₋₇ carbocyclyl or C₂₋₄ alkenyl group.

14. A compound according to any one of the preceding claims which is: (a) a thiophene carboxamide derivative of formula (IC) or (ID), or a tautomer thereof; or (b) a pharmaceutically acceptable salt, N-oxide, hydrate or solvate thereof: 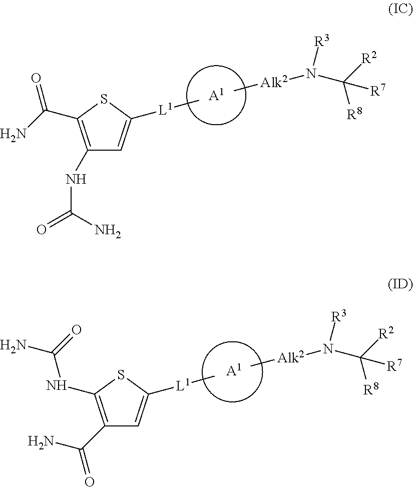
wherein:
L¹ represents unsubstituted C₁₋₄ alkylene;
A¹ represents 1,4-phenylene or 1,3-phenylene, which is unsubstituted or substituted by 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl;
Alk² represents an unsubstituted C₁₋₃ alkylene, C₂₋₃ alkenylene or C₂₋₃ alkynylene group;
R² represents —COOR⁹ wherein R⁹ represents an unsubstituted C₁₋₄ alkyl, C₃₋₇ carbocyclyl or C₂₋₄ alkenyl group;
R³ represents a hydrogen atom or an unsubstituted methyl group; and
R⁷ and R⁸, which are the same or different, represent a hydrogen atom or an unsubstituted C₁₋₆ alkyl group.

15. A compound according to claim 14, wherein R⁸ represents a hydrogen atom and which has the formula (IC′) or (ID′):

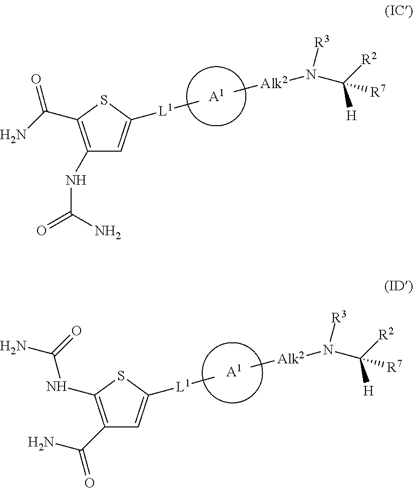

16. A compound as claimed in claim 14 wherein:
A¹ represents 1,3-phenylene, which is unsubstituted or substituted by 1, 2 or 3 substituents which are the same or different and are selected from halogen atoms and unsubstituted C₁₋₄ alkyl, C₁₋₄ alkoxy, hydroxyl and —NR′R″ groups wherein R′ and R″ are the same or different and represent hydrogen or unsubstituted C₁₋₂ alkyl; and
R⁷ and R⁸, which are the same or different, represent an unsubstituted C₁₋₆ alkyl group.

17. A compound as claimed in any one of the preceding claims which is selected from:
Cyclopentyl 2-(4-((4-carbamoyl-5-ureidothiophen-2-yl)methyl)benzylamino)-4-methylpentanoate;
Tert-butyl 2-(4-((4-carbamoyl-5-ureidothiophen-2-yl)methyl)benzylamino)-4-methylpentanoate;
Cyclopentyl 2-(4-((4-carbamoyl-5-ureidothiophen-2-yl)methyl)benzylamino)-4-methylpentanoate; and
Tert-butyl 2-(4-((4-carbamoyl-5-ureidothiophen-2-yl)methyl)benzylamino)-4-methylpentanoate.

18. A compound as defined in any one of the preceding claims, for use in a method of treatment of the human or animal body.

19. A pharmaceutical composition which comprises a compound as defined in any one of claims 1 to 17 and a pharmaceutically acceptable carrier or diluent.

20. A compound as defined in any one of claims 1 to 17 for use in the treatment of a disorder mediated by an IKK enzyme, wherein said IKK enzyme is preferably IKK-3.

21. A compound for use in the treatment of a disorder as claimed in claim 20, wherein said disorder is selected from neoplastic/proliferative, immune and inflammatory diseases.

22. A compound for use in the treatment of a disorder as claimed in claim 20, wherein said disorder is cancer.

23. A compound for use in the treatment of a disorder as claimed in claim 20, wherein said disorder is selected from: bowel cancer; ovarian cancer; head, neck or cervical squamous cancer; gastric or lung cancer; anaplastic oligodendrogliomas; glioblastoma multiforme; or medulloblastomas.

24. A compound for use in the treatment of a disorder as claimed in claim 20, wherein said disorder is selected from: rheumatoid arthritis, psoriasis, inflammatory bowel disease, Crohn's disease, ulcerative colitis, chronic obstructive pulmonary disease, asthma, multiple sclerosis, diabetes, atopic dermatitis, graft versus host disease, systemic lupus erythematosus, Type II diabetes mellitus or Alzheimers disease.

25. A method of treating or preventing a disorder mediated by IKK in a patient, which method comprises administering to said patient an effective amount of a compound as defined in any one of claims 1 to 17.

26. Use of a compound as defined in any one of claims 1 to 17 in the manufacture of a medicament for use in the treatment or prevention of a disorder mediated by an IKK enzyme, wherein said IKK enzyme is preferably IKK-β.
